(19) United States
(12) Patent Application Publication
(10) Pub. No.: US 2024/0000951 A1
(43) Pub. Date: Jan. 4, 2024

(54) GPX4 PROTEIN DEGRADATION-INDUCING COMPOUND

(71) Applicant: UPPTHERA, Incheon (KR)

(72) Inventors: Soo Hee Ryu, Incheon (KR); Ji Hoon Ryu, Seoul (KR); Hwa Jin Lee, Incheon (KR); Im Suk Min, Gyeonggi-do (KR); Han Kyu Lee, Gyeonggi-do (KR); Young Jin Choi, Incheon (KR); Seong Hoon Kim, Incheon (KR)

(73) Assignee: UPPTHERA, Incheon (KR)

(21) Appl. No.: 18/039,895

(22) PCT Filed: Dec. 2, 2021
(86) PCT No.: PCT/KR2021/018161

§ 371 Date: Jun. 1, 2023

(30) Foreign Application Priority Data
Dec. 3, 2020 (KR) 10-2020-0167725
Jun. 30, 2021 (KR) 10-2021-0085982

(51) Int. Cl. A61K 47/55; A61K 47/54; A61P 35/00

(52) U.S. Cl. CPC A61K 47/55; A61P 35/00; A61K 47/545

(57) ABSTRACT

The present invention relates to a GPX4 protein degradation-inducing compound. Specifically, the present invention provides a bifunctional compound in which a GPX4 protein-binding moiety and a CRBN E3 ubiquitin ligase-binding moiety are linked by a chemical linker, a method for preparing the same, a method for degrading a GPX4 protein using the same, and the use for preventing or treating GPX4-related diseases or ferroptosis-related diseases.

### TECHNICAL FIELD

The present invention relates to a GPX4 protein degradation-inducing compound, a method for preparing the same, and use for preventing or treating GPX4-related diseases using the same.

### BACKGROUND ART

Ferroptosis is a type of programmed cell death induced in an iron-dependent manner by accumulation of lipid peroxidation in cells. The programmed cell death caused by the ferroptosis pathway in cells is inhibited by the antioxidant enzyme activity of glutathione peroxidase 4 (GPX 4).

GPX4, a subtype of the glutathione peroxidase (GPX) family, reduces lipid peroxides in cell membranes to lipid-alcohols, while simultaneously catalyzing the disulfide reaction of two molecules of glutathione, a cofactor. In the above reaction, selenocysteine, located at the center of the enzyme activity of GPX4, plays a key role.

It is known that inhibition of the activity of GPX4 may cause cell death by ferroptosis by inhibiting the lipid peroxide removal function in cells. GPX4 protein has attracted attention as a drug target for various diseases, including treatment-resistant cancer, where therapeutic effects are expected by ferroptosis induction, and accordingly, various small-molecule GPX4 inhibitors such as RSL3, DPI10, DPI7, ML162, and ML210, and the like, have been developed to date (document[Yang, Wan Seok, et al. “Regulation of ferroptotic cancer cell death by GPX4.” Cell 156.1-2 (2014): 317-331.], document[Li, Jie, et al. “Ferroptosis: past, present and future.” Cell death & disease 11.2 (2020): 1-13.], and the like).

However, GPX4 protein has a structure that makes it difficult for small molecules to bind to the central site of a key enzyme, having difficulties in developing small molecule inhibitors. For example, GPX4 inhibitors such as RSL3 covalently bonded to GPX4 through a reactive alkyl chloride moiety have been consistently reported to have a wide range of off-target side effects and pharmacokinetic disadvantages, and the like (document[Eaton, John K., et al. “Selective covalent targeting of GPX4 using masked nitrile-oxide electrophiles.” Nature chemical biology 16.5 (2020): 497-506.]). Therefore, for the development of a therapeutic agent capable of inducing ferroptosis by inhibiting the activity of GPX4, it is required to develop alternative therapeutic drugs to solve the problems encountered in the development of conventional small molecule compounds.

### DISCLOSURE

### Technical Problem

An object of the present invention is to provide a novel GPX4 protein degradation-inducing compound.

Another object of the present invention is to provide a method for preparing a GPX4 protein degradation-inducing compound and use thereof.

### Technical Solution

The present inventors newly synthesized a proteolysis-targeting chimera (PROTAC) compound targeting the degradation of GPX4 protein, and first discovered that the compound could effectively degrade GPX4 protein in cells to be used for the treatment of GPX4-related diseases. Accordingly, the present invention provides a GPX4 protein degradation-inducing compound, a method for preparing the same, and use thereof.

GPX4 Protein Degradation-Inducing Compound

In one aspect, the present invention provides a novel compound that induces degradation of GPX4 protein. Specifically, the present inventors provide a GPX4 protein targeting degradation-inducing compound utilizing CRBN E3 ubiquitin ligase.

In an embodiment, the present invention provides a compound represented by the following Formula I, a stereoisomer thereof, or a pharmaceutically acceptable salt thereof:

ULM-Linker-GTM [Formula I]

in Formula I above, GTM is a GPX4 protein binding moiety, ULM is a CRBN E3 ubiquitin ligase binding moiety, and Linker is a group chemically linking ULM and GTM.

The present inventors found for the first time that the compound represented by the Formula I could place the GPX4 protein and the E3 ubiquitin ligase in close proximity within cells, thereby inducing artificial ubiquitination of the GPX4 protein, and thus degradation of the GPX4 protein could be achieved by an ubiquitin-proteasome system (UPS) in the cells (FIG. 1).

In other words, the technical feature of the present invention is the first discovery of the GPX4 protein-targeting PROTAC (proteolysis targeting chimera) compound and usefulness thereof.

The compound represented by Formula I, which is GPX4 protein-targeting PROTAC compound of the present invention, is composed of ULM, GTM, and Linker moieties, and each moiety will be described below.

(1) GPX4 Protein Binding Moiety (GTM)

In the compound represented by Formula I of the present invention, GTM is a GPX4 protein binding moiety.

In the present invention, GPX4 (Glutathione peroxidase 4) protein is a protein encoded by GPX4 gene (GPX4 glutathione peroxidase 4 [Homo sapiens (human)], Gene ID: 2879). GPX4 protein is an antioxidant enzyme that reduces lipid peroxides in cells and plays a role in the protection of the cells against oxidative stress. GPX4 protein has selenocysteine located at the center of enzyme activity, and performs a reaction of reducing lipid peroxides to lipid-alcohols by using glutathione as a cofactor. It is known that inhibition of the activity of GPX4 protein is able to induce artificial ferroptosis inhibition, which may be utilized for the treatment of various diseases such as treatment-resistant cancer, and the like.

In the Formula I of the present invention, GTM may have a compound structure capable of inhibiting the activity of GPX4 protein by directly binding to the GPX4 protein, wherein the compound is known in the art to which the present invention belongs.

As an example, an inhibitor compound that binds directly to GPX4 protein may be referred to as having the following structure (document[Wang, Liyuan, Xiaoguang Chen, and Chunhong Yan. “Ferroptosis: An Emerging Therapeutic Opportunity for Cancer.” Genes & Diseases (2020).], and the like).

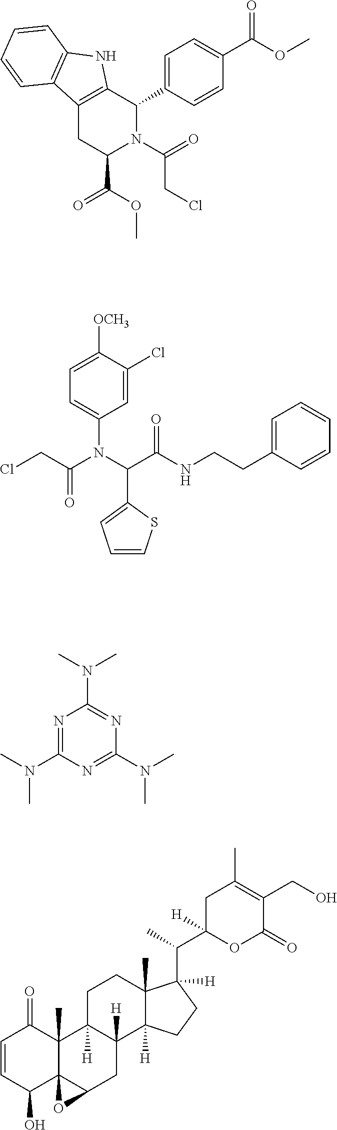

As another example, an inhibitor compound that binds directly to GPX4 protein may be referred to as having the following structure (document[Eaton, John K., et al. “Selective covalent targeting of GPX4 using masked nitrile-oxide electrophiles.” Nature chemical biology 16.5 (2020): 497-506.] ‘Supplementary FIG. 1’, and the like).

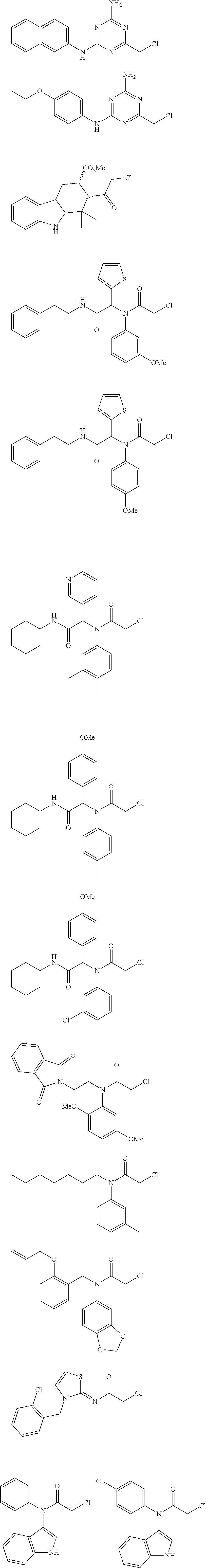

The compound represented by Formula I of the present invention functions to degrade GPX4 protein by inducing ubiquitination of GPX4 protein in cells through the bifunctionality of the PROTAC compound. The GTM moiety of Formula I of the present invention is functionally distinct from the GPX4 inhibitor as a single compound, but those skilled in the art, when referring to the specification of the present invention, may appropriately select and synthesize a chemical structure capable of being used as the GTM moiety of Formula I of the present invention, based on the GPX4 protein binding ability according to the GPX4 inhibitor structure known in the art and the technical idea presented in the present invention.

In an embodiment, the GTM of Formula I of the present invention is represented by the following Formula G-1.

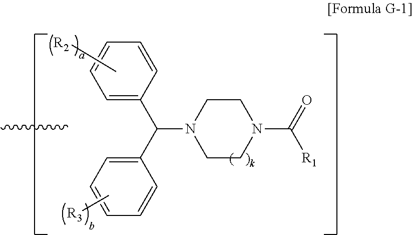

in Formula G-1 above,

R₁, R₂ and R₃ are each independently hydrogen, halogen, OH, NH₃, NO₂, CN, alkyl, alkene, alkyne, cycloalkyl, heterocyclyl, aryl or heteroaryl {wherein at least one hydrogen in R₁, R₂ and R₃ may each independently be optionally substituted};

k, a, and b are each independently an integer of 1 to 3;

[symbol] represents that any one hydrogen or halogen in the Formula G-1 is substituted with a single bond and connected to the Linker by a covalent bond.

As an example, the GPX4 protein-binding compound having the structure of Formula G-1 is as follows, and a preparation method thereof and GPX4 protein-binding ability are described in document[Eaton, John K., et al. “Structure-activity relationships of GPX4 inhibitor warheads.” Bioorganic & Medicinal Chemistry Letters 30.23 (2020): 127538.], and the like.

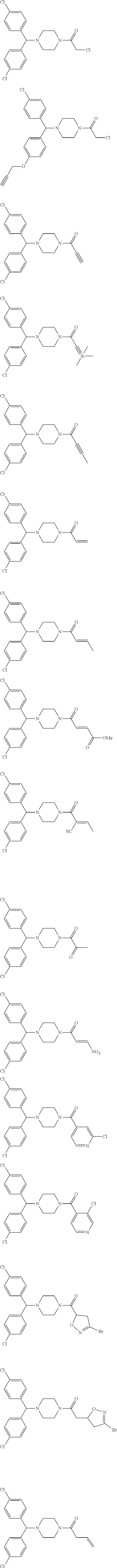

In the compound represented by Formula I of the present invention, the Formula G-1 moiety is covalently linked to the Linker via [symbol] . Here, any hydrogen or halogen in the G-1 moiety structure may be substituted with a single bond to be connected to the Linker. For example, the G-1 moiety may be connected to the Linker by substituting hydrogen or halogen of the R₂ group in the G-1 moiety with a single bond.

More specifically, the Formula G-1 may be a GTM protein binding moiety that satisfies the following definitions:
{R₁ is selected from the group consisting of

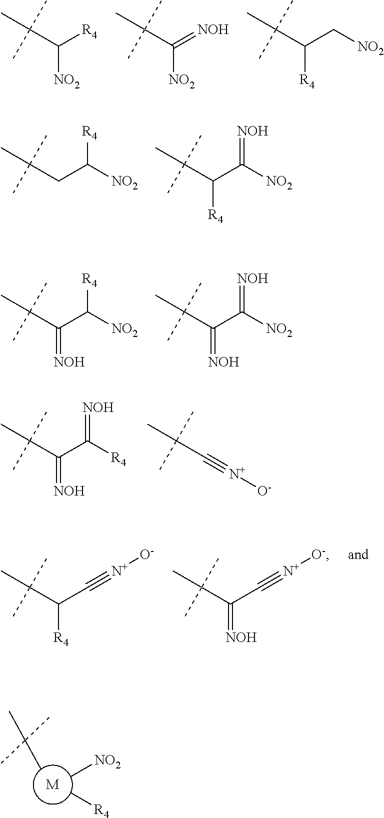

{wherein

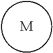

is 3-7 membered cycloalkyl, 4-8 membered heterocyclyl, phenyl or 4-8 membered heteroaryl};
R₂ and R₃ are each independently hydrogen, halogen, OH, NH₃, NO₂, CN, C₁₋₆alkyl, C₁₋₆alkoxy, C₂₋₆ alkenyl or C₂₋₆ alkynyl {wherein the C₁₋₆alkyl, C₁₋₆alkoxy, C₂₋₆ alkenyl or C₂₋₆ alkynyl may be substituted with 1 to 3 halogens, OH, NH₃, NO₂ or CN};
R₄ is —(C₀₋₄ alkylene)-R₅, (C₀₋₄ alkylene)-R_{L1}—(C₀₋₄alkylene)-R₅ or —(C₀₋₄ alkylene)-R_{L1}—(C₀₋₄alkylene)-R_{L2}—(C₀₋₄alkylene)-R₅ {wherein at least one hydrogen in R₄ may each independently be substituted with halogen, C₁₋₃ alkyl, C₁₋₃ alkoxy, OH, NH₂, NO₂ or CN};
R₅ is hydrogen, halogen, OH, OCH₃, COH, COOH, CN, NH₂, NH (C₁₋₃ alkyl), NCH₃(C₁₋₃alkyl), SO₂, C₁₋₆alkyl, C₂₋₆alkenyl, C₂₋₆alkynyl, C₃₋₈ cycloalkyl, 4-8 membered heterocyclyl, phenyl or 4-8 membered heteroaryl;
R_{L1} and R_{L2} are each independently —O—, —OO—, —COO—, —OCO—, —NH—, —N(C₁₋₃ alkyl)-, —NHCO—, —N(C₁₋₃ alkyl)CO—, —CONH—, —CON (C₁₋₃ alkyl)- or —NHCONH—;
a, b and k are each independently 1 or 2; and
[symbol] represents that any one hydrogen or halogen in the Formula G-1 is substituted with a single bond and connected to the Linker by a covalent bond}.

The GPX4 protein-binding compound of the Formula G-1 structure having the above substituents is as follows, and a preparation method thereof and GPX4 protein-binding ability are known in the document[Weiwer, Michel, et al. “Development of small-molecule probes that selectively kill cells induced to express mutant RAS.” Bioorganic & medicinal chemistry letters 22.4 (2012): 1822-1826.], and document[Eaton, John K., et al. “Selective covalent targeting of GPX4 using masked nitrile-oxide electrophiles.” Nature chemical biology 16.5 (2020): 497-506.], and the like.

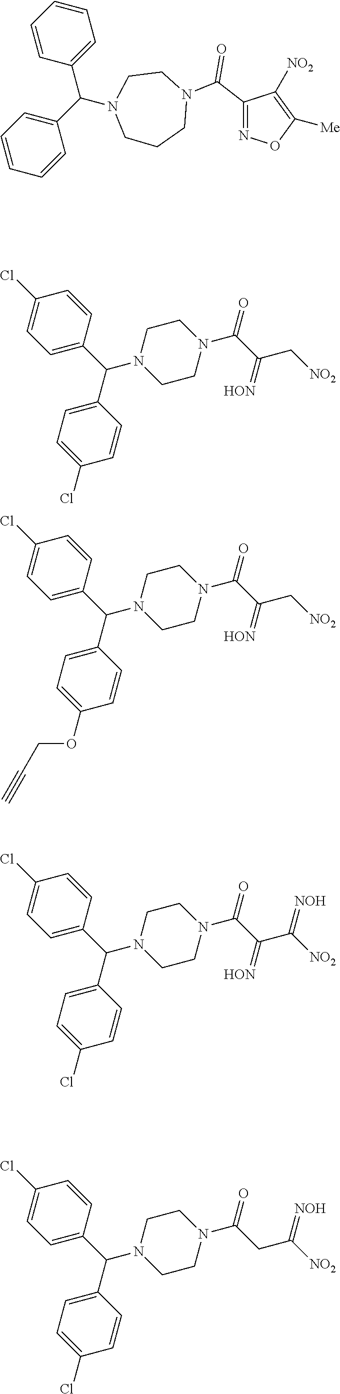

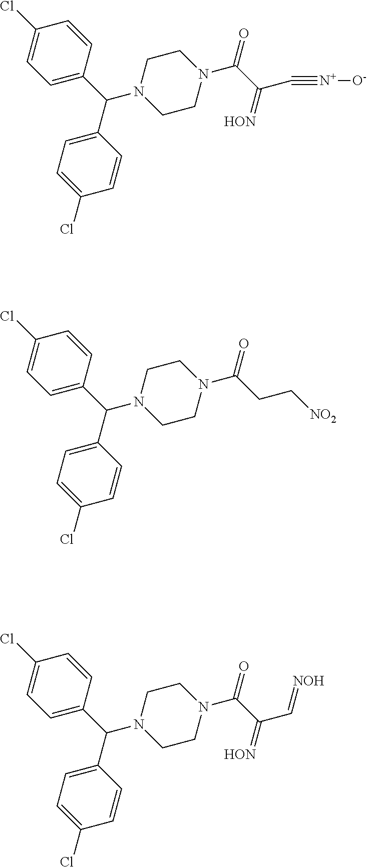

In a more specific embodiment, the Formula G-1 above is represented by the following Formula G-2:

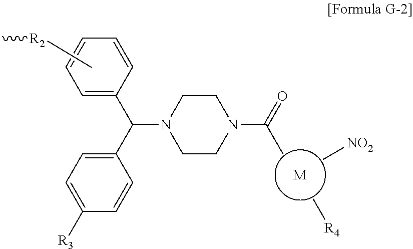

in Formula G-2 above,

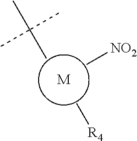

is selected from the group consisting of

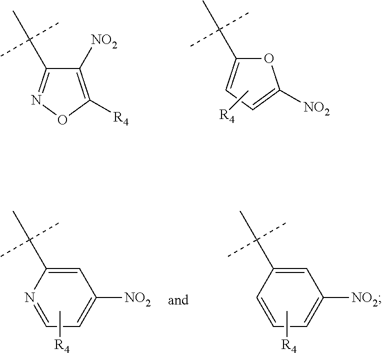

R₂ and R₃ are each independently hydrogen, halogen, OH, NH₃, C₁₋₆alkyl, C₁₋₆alkoxy {wherein the C₁₋₆alkyl or C₁₋₆ alkoxy may be substituted with one halogen or CN};
hydrogen or halogen in R₂ is substituted with [symbol] {wherein [symbol] represents that it is linked to the Linker by a covalent bond};
R₄ is —(C₀₋₂ alkylene)-R₅ or —(C₀₋₂ alkylene)-R_{L1}—(C₀₋₂alkylene)-R₅ {wherein one hydrogen in R₄ may be substituted by halogen or OH};
R₅ is hydrogen, halogen, COOH, C₁₋₆ alkyl, C₂₋₆ alkenyl, C₅₋₆cycloalkyl, 5-6 membered heterocyclyl, phenyl or 5-6 membered heteroaryl; and
R_{L1} is —O—, —CO—, —COO—, —OCO—, —NHCO— or —CONH—.

As an example, the GPX4 protein-binding compound having the structure of Formula G-2 above is as follows, and a preparation method thereof and GPX4 protein-binding ability are described in document[Weiwer, Michel, et al. “Development of small-molecule probes that selectively kill cells induced to express mutant RAS.” Bioorganic & medicinal chemistry letters 22.4 (2012): 1822-1826.], and the like.

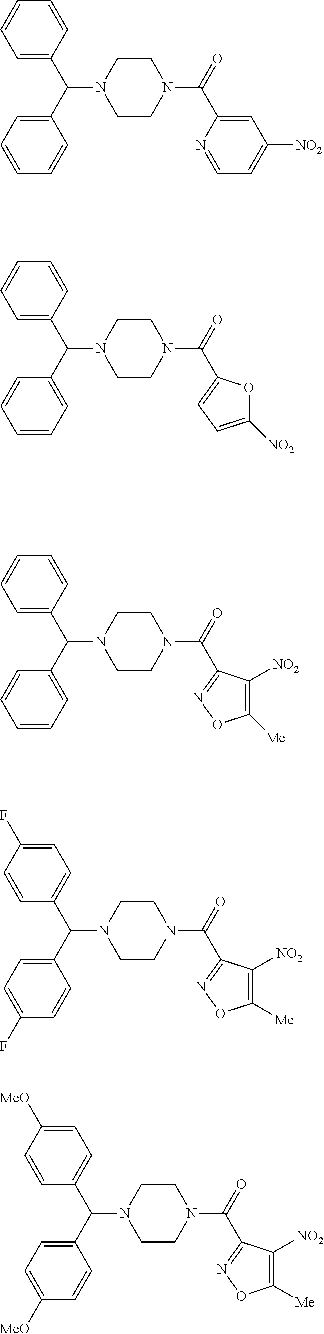 [SMILES: O=C(c1cc([N+](=O)[O-])ccn1)N1CCN(C(c2ccccc2)c2ccccc2)CC1 ; O=C(c1ccc([N+](=O)[O-])o1)N1CCN(C(c2ccccc2)c2ccccc2)CC1 ; Cc1onc(C(=O)N2CCN(C(c3ccccc3)c3ccccc3)CC2)c1[N+](=O)[O-] ; Cc1onc(C(=O)N2CCN(C(c3ccc(F)cc3)c3ccc(F)cc3)CC2)c1[N+](=O)[O-] ; COc1ccc(C(c2ccc(OC)cc2)N2CCN(C(=O)c3noc(C)c3[N+](=O)[O-])CC2)cc1]

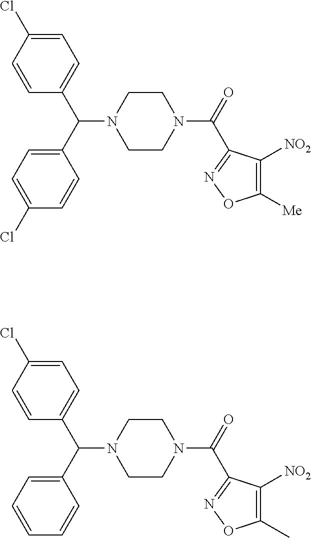 [SMILES: Cc1onc(C(=O)N2CCN(C(c3ccc(Cl)cc3)c3ccc(Cl)cc3)CC2)c1[N+](=O)[O-] ; Cc1onc(C(=O)N2CCN(C(c3ccccc3)c3ccc(Cl)cc3)CC2)c1[N+](=O)[O-]]

As another example, the GPX4 protein-binding compound having the structure of Formula G-2 above is as follows, and a preparation method thereof and GPX4 protein-binding ability are described in document[Eaton, John K., et al. “Selective covalent targeting of GPX4 using masked nitrile-oxide electrophiles.” Nature chemical biology 16.5 (2020): 497-506.], and the like.

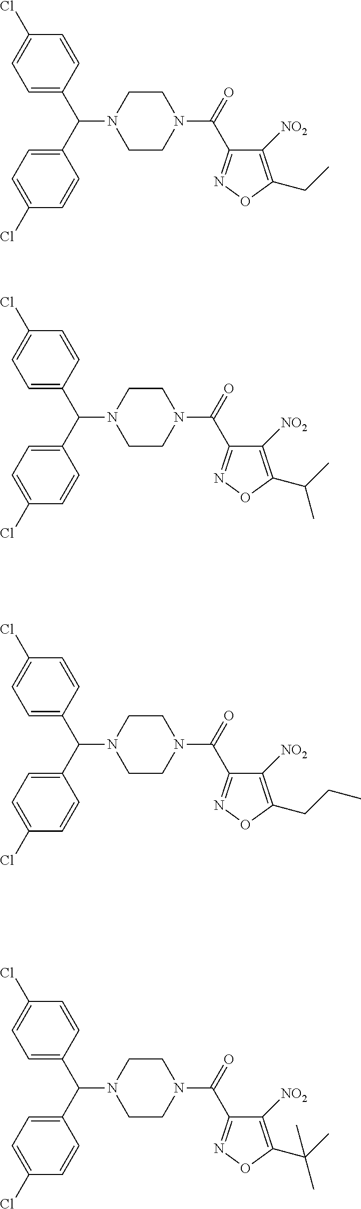 [SMILES: CCc1onc(C(=O)N2CCN(C(c3ccc(Cl)cc3)c3ccc(Cl)cc3)CC2)c1[N+](=O)[O-] ; CC(C)c1onc(C(=O)N2CCN(C(c3ccc(Cl)cc3)c3ccc(Cl)cc3)CC2)c1[N+](=O)[O-] ; CCCc1onc(C(=O)N2CCN(C(c3ccc(Cl)cc3)c3ccc(Cl)cc3)CC2)c1[N+](=O)[O-] ; CC(C)(C)c1onc(C(=O)N2CCN(C(c3ccc(Cl)cc3)c3ccc(Cl)cc3)CC2)c1[N+](=O)[O-]]

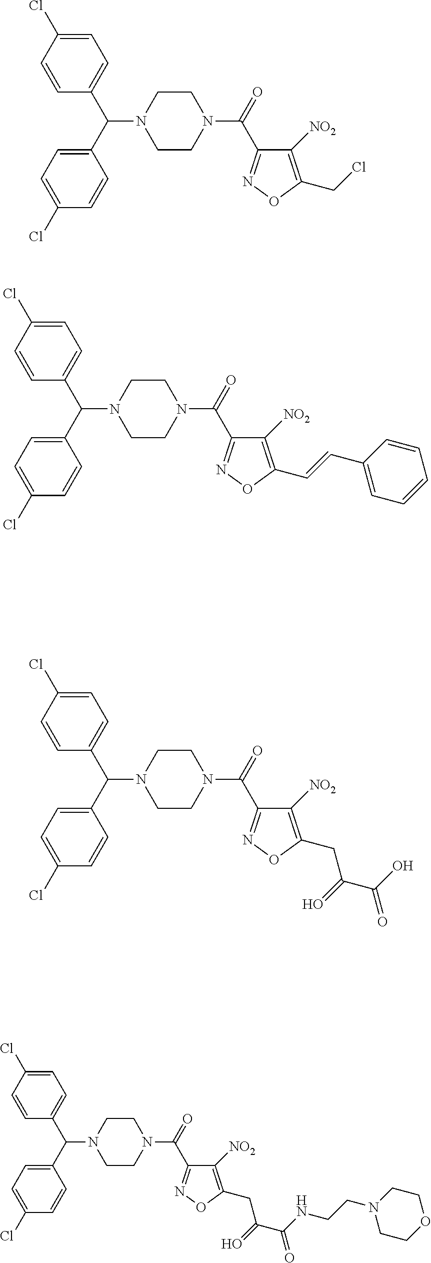 [SMILES: O=C(c1noc(CCl)c1[N+](=O)[O-])N1CCN(C(c2ccc(Cl)cc2)c2ccc(Cl)cc2)CC1 ; O=C(c1noc(/C=C/c2ccccc2)c1[N+](=O)[O-])N1CCN(C(c2ccc(Cl)cc2)c2ccc(Cl)cc2)CC1 ; O=C(O)C(=O)Cc1onc(C(=O)N2CCN(C(c3ccc(Cl)cc3)c3ccc(Cl)cc3)CC2)c1[N+](=O)[O-] ; O=C(Cc1onc(C(=O)N2CCN(C(c3ccc(Cl)cc3)c3ccc(Cl)cc3)CC2)c1[N+](=O)[O-])C(=O)NCCN1CCOCC1]

As still another example, the GPX4 protein-binding compound having the structure of Formula G-2 above is as follows, and a preparation method thereof and GPX4 protein-binding ability are described in document[Eaton, John K., et al. “Structure-activity relationships of GPX4 inhibitor warheads.” Bioorganic & Medicinal Chemistry Letters 30.23 (2020): 127538.], and the like.

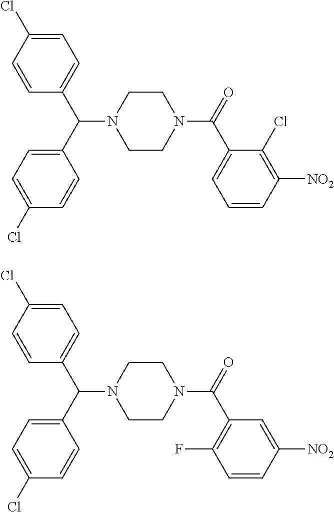

As in Formula G-1, the Formula G-2 moiety is covalently linked to the Linker via [symbol] . Here, any hydrogen or halogen in the G-2 moiety structure may be substituted with a single bond to be connected to the Linker. For example, the G-2 moiety may be connected to the Linker by substituting hydrogen or halogen of the R₂ group in the G-2 moiety with a single bond.

(2) CRBN E3 Ubiquitin Ligase Binding Moiety (ULM)

In the present invention, E3 ubiquitin ligase is a protein that promotes the ubiquitin transfer to a target substrate protein, and ULM in the compound represented by Formula I according to the present invention is a moiety capable of binding to CRBN E3 ubiquitin ligase.

In the present invention, CRBN refers to Cereblon E3 ubiquitin ligase. CRBN together with DDB1, Cul4A, and ROC1 constitute an E3 ubiquitin ligase complex, wherein CRBN is a substrate recognition subunit of the complex. Some compounds capable of binding to CRBN E3 ubiquitin ligase are known in the art.

For example, since it was known that thalidomide binds to the CRBN E3 ubiquitin ligase (document[Ito et al. 2010]), a number of imide-based small molecule compounds (immunomodulatory imide drug; IMiD) including lenalidomide and pomalidomide have been reported to have CRBN binding ability (document[Chamberlain and Brian. 2019], document[Akuffo et al. 2018]), document[Burslem et al. 2018], and the like). In addition, the binding structure of thalidomide, which is a representative CRBN E3 ubiquitin ligase binding material, and the CRBN E3 ubiquitin ligase is known in the art as shown in FIG. 3 (PDB code: 4CI1), which allows the skilled person to appropriately select a structure capable of functioning as the CRBN E3 ubiquitin ligase binding moiety (ULM) in Formula I.

In an embodiment, the CRBN E3 ubiquitin ligase binding moiety of the present invention is a compound represented by the following Formula A-1:

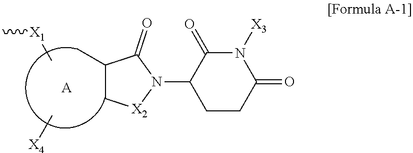

in Formula A-1 above,

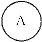

is a ring selected from the group consisting of

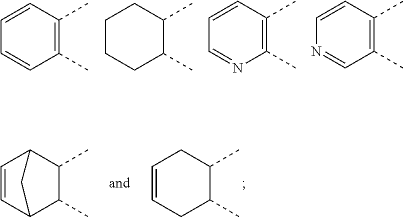

X₁ is a single bond, —CH₂—, —NH—, —O—, —CH₂CH₂—, —CC— —CO—, —OCO—, —NHCO— or —CONH—;
X₂ is —CH₂—, —CH (C₁₋₄alkyl)-, —NH—, —N(C₁₋₄ alkyl)-, —O—, —CO—, —CH₂—CH₂—, —NH—CH₂—, —NH—CH(C₁₋₄ alkyl)-, —N═CH—, N═C (C₁₋₄alkyl)- or —N═N—;
X₃ is hydrogen or C₁₋₄ alkyl; and
X₄ is hydrogen, halogen, C₁₋₆ alkyl, CN, NH₂, NO₂, OH, COH, COOH or CF₃.

In an embodiment, the Formula A-1 above is represented by the following Formula A-2:

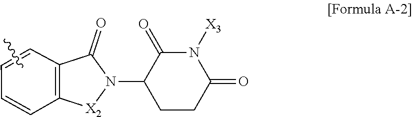

in Formula A-2 above,
X₂ is —CH₂—, —CH(C₁₋₄ alkyl)-, —CO— or —N═N—; and
X₃ is hydrogen or C₁₋₃ alkyl.

In an embodiment of the present invention, the Formula A-2 above may be selected from the group consisting of the following moieties.

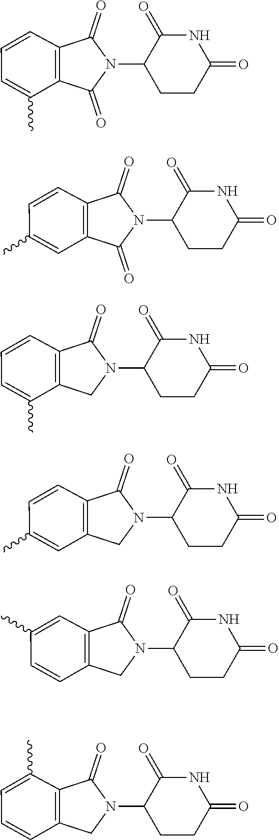

An example of the CRBN E3 ubiquitin ligase binding moiety according to the present invention is as follows (document[Chamberlain and Brian. 2019] and document[Akuffo et al. 2018]).

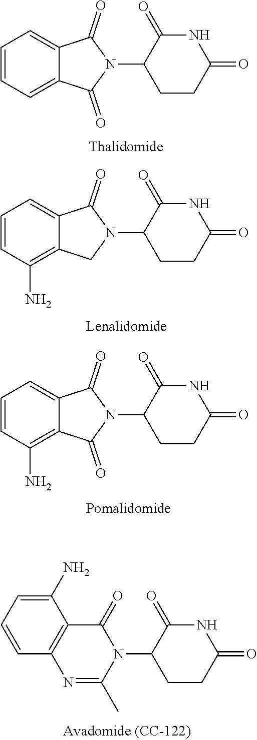 [SMILES: O=C1CCC(N2C(=O)c3ccccc3C2=O)C(=O)N1 ; Nc1cccc2c1CN(C1CCC(=O)NC1=O)C2=O ; Nc1cccc2c1C(=O)N(C1CCC(=O)NC1=O)C2=O ; Cc1nc2cccc(N)c2c(=O)n1C1CCC(=O)NC1=O]

Another example of the CRBN E3 ubiquitin ligase binding moiety according to the present invention is as follows (document[Burslem et al. 2018]).

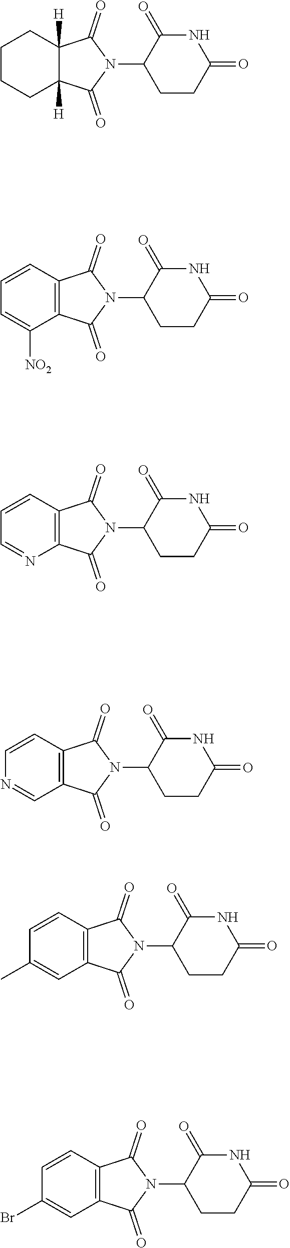 [SMILES: O=C1CCC(N2C(=O)[C@H]3CCCC[C@H]3C2=O)C(=O)N1 ; O=C1CCC(N2C(=O)c3cccc([N+](=O)[O-])c3C2=O)C(=O)N1 ; O=C1CCC(N2C(=O)c3cccnc3C2=O)C(=O)N1 ; O=C1CCC(N2C(=O)c3ccncc3C2=O)C(=O)N1 ; Cc1ccc2c(c1)C(=O)N(C1CCC(=O)NC1=O)C2=O ; O=C1CCC(N2C(=O)c3ccc(Br)cc3C2=O)C(=O)N1]

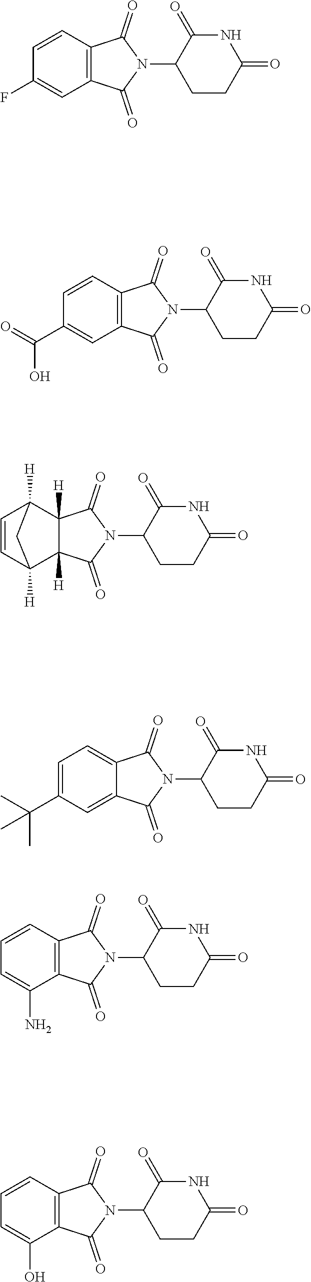 [SMILES: O=C1CCC(N2C(=O)c3ccc(F)cc3C2=O)C(=O)N1 ; O=C1CCC(N2C(=O)c3ccc(C(=O)O)cc3C2=O)C(=O)N1 ; O=C1CCC(N2C(=O)[C@@H]3[C@H](C2=O)[C@H]2C=C[C@@H]3C2)C(=O)N1 ; CC(C)(C)c1ccc2c(c1)C(=O)N(C1CCC(=O)NC1=O)C2=O ; Nc1cccc2c1C(=O)N(C1CCC(=O)NC1=O)C2=O ; O=C1CCC(N2C(=O)c3cccc(O)c3C2=O)C(=O)N1]

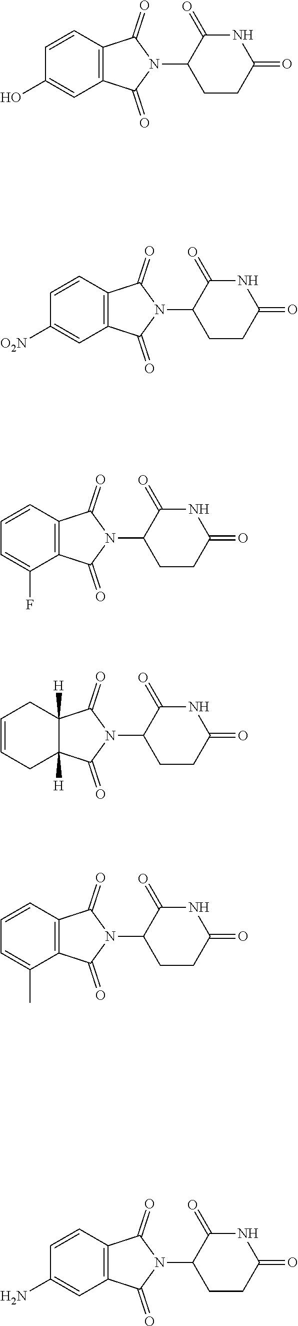 [SMILES: O=C1CCC(N2C(=O)c3ccc(O)cc3C2=O)C(=O)N1 ; O=C1CCC(N2C(=O)c3ccc([N+](=O)[O-])cc3C2=O)C(=O)N1 ; O=C1CCC(N2C(=O)c3cccc(F)c3C2=O)C(=O)N1 ; O=C1CCC(N2C(=O)[C@H]3CC=CC[C@H]3C2=O)C(=O)N1 ; Cc1cccc2c1C(=O)N(C1CCC(=O)NC1=O)C2=O ; Nc1ccc2c(c1)C(=O)N(C1CCC(=O)NC1=O)C2=O]

(3) Linker

In the compound represented by Formula I of the present invention, Linker is a group that chemically connects the ULM and GTM moieties. Through the Linker, the E3 ubiquitin ligase protein targeted by the ULM moiety and the GPX4 protein targeted by the GTM moiety may interact with each other within an appropriate physical distance, thereby inducing ubiquitination of the target GPX4 protein. Accordingly, the Linker is included in the present invention without limitation as long as it is a chemical group in the form in which the compound represented by Formula I of the present invention is capable of performing the function of PROTAC compounds that induce ubiquitination of the target GPX4 protein.

In an embodiment, the Linker is represented by the following Formula L:

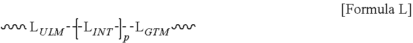

in Formula L above, [symbol] and [symbol] are bonds,
L_{ULM} binds to the ULM moiety through [symbol] linked thereto,
L_{GTM} binds to the GTM moiety through [symbol] linked thereto,
L_{ULM}, L_{GTM}, and L_{INT} are each independently selected from the group consisting of a single bond, —CH₂—, —NH—, —O—, —S—, —SO—, —SO₂—, —CO—, —CH₂CH₂—, —CHCH—, —CC—, —CH₂CH₂O—, —OCH₂CH₂—, —CH₂CH₂S—, —SCH₂CH₂—, —COO—, —CONH—, —NHCO—, and

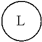

{wherein

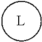

is cycloalkyl, heterocyclyl, aryl or heteroaryl};
L_{ULM}, L_{GTM}, and L_{INT} may each independently be substituted with at least one C₁₋₆ alkyl, C₃₋₈ cycloalkyl, halogen, hydroxy, amine, nitro, cyano or haloalkyl; and
p is an integer from 1 to 30.

In an embodiment, p is an integer of 1 or more, 5 or more, 10 or more, 15 or more, 20 or more, or 25 or more, and is an integer of 25 or less, 20 or less, 15 or less, 10 or less, or 5 or less.

In the present invention, L_{ULM} may be

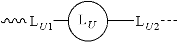

{wherein L_{U1} is selected from the group consisting of a single bond, —CH₂—, —CH₂CH₂—, —CH═CH—, —CC—, —NH—, —NCH₃—, —CO—, —NHCO—, and —O—;
L_{U2} is selected from the group consisting of a single bond, —CH₂—, —NH—, —O—, —CO—, and —CONH—; and

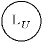

is a single bond or selected from the group consisting of C₁₋₆alkyl, 3-10 membered cycloalkyl, 4-10 membered heterocyclyl, 6-10 membered aryl or 5-10 membered heteroaryl}.

In the present invention, L_{GTM} may be

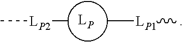

{wherein L_{P1} is selected from the group consisting of a single bond, —O—, —S—, —NH—, —N(C₁₋₄ alkyl)-, —CH₂—, —CH(C₁₋₄alkyl)-, —CH₂NH—, and —CH₂CH₂—,
L_{P2} is selected from the group consisting of a single bond, —CO—, —COCH₂—, —NHCO—, —NHCOCH₂—, —HET-, and -HET-CH₂—, wherein HET is a 5-6 membered heterocyclyl or heteroaryl having at least one N, S or O atom, and

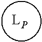

is a single bond, C₁₋₈ alkyl substituted with amine group; or a ring selected from the group consisting of 3-10 membered cycloalkyl, 4-10 membered heterocyclyl, 6-10 membered aryl, and 5-10 membered heteroaryl.}. In an embodiment of the present invention,

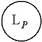

may be

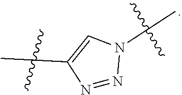 [SMILES: *c1cn(*)nn1]

In the present invention, may be

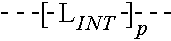

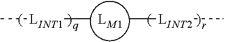

{wherein

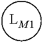

is a single bond; or a ring selected from the group consisting of 3-10 membered cycloalkyl, 4-10 membered heterocyclyl, 6-10 membered aryl and 5-10 membered heteroaryl,

L_{INT1} and L_{INT2} are each independently selected from the group consisting of —CH₂—, —NH—, —NCH₃—, —O—, —S—, —SO—, —SO₂—, —CO—, —CH₂CH₂O—, —OCH₂CH₂—, —CH₂CH₂S—, —SCH₂CH₂—, —COO—, —CONH—, and —NHCO—, and
q and r are each independently an integer of 1 to 10}.

In an embodiment, the Linker is a Linker contained in the PROTAC compounds provided in Examples 1 to 5 of the present invention.

According to a specific embodiment of the present invention, the compound represented by Formula I is Compounds 1 to 5 provided in Examples 1 to 5 below, a stereoisomer thereof or a pharmaceutically acceptable salt thereof.

In the present invention, the term “compound” also includes, in addition to single compounds, tautomers, optical isomers (including racemic mixtures), specific enantiomers, or enantiomerically enriched mixtures. The substituent terms used to describe the structures of the compounds of the present invention have the same meanings as commonly used in the field of organic chemistry.

In the present invention, a pharmaceutically acceptable salt refers to any organic acid or inorganic acid addition salt at a concentration having a relatively non-toxic and harmless effective action to patients, in which side effects caused by this salt does not reduce the beneficial efficacy of the compound represented by Formula I. For example, the pharmaceutically acceptable salt may be an inorganic acid such as hydrochloric acid, phosphoric acid, sulfuric acid, nitric acid, or the like, or an organic acid such as methanesulfonic acid, p-toluenesulfonic acid, formic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, or hydroiodic acid, but is not limited thereto.

Method for Preparing GPX4 Protein Degradation-Inducing Compound

The compound represented by Formula I of the present invention, a stereoisomer thereof or a pharmaceutically acceptable salt thereof may be prepared through reactions such as the following Reaction Schemes 1 to 3 by synthetic methods known in the technical field of organic chemistry or modification and derivatization techniques obvious to those skilled in the art:

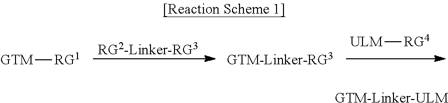

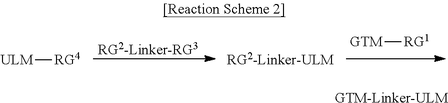

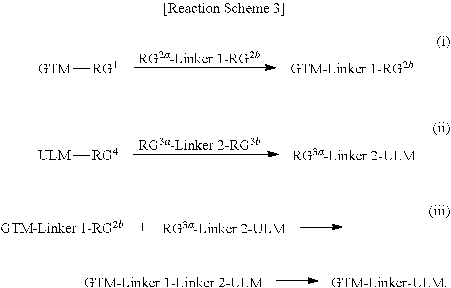

In Reaction Schemes 1 to 3 above, GTM, Linker, and ULM are the above-defined groups or reaction derivatives thereof, and RG¹, RG², RG^{2a}, RG^{2b}, RG³, RG^{3a}, RG^{3b}, and RG⁴ are moieties containing suitable reactive groups capable of linking together with the PROTAC compound intermediate represented by Formula I through covalent bond formation in the field of organic synthesis. The covalent bond formation may be performed through synthesis reactions such as amide formation, ester formation, carbamate formation, urea formation, ether formation, amine formation, and single bonds, double bonds formation between various carbons, click chemistry, and the like, depending on the specific reactive group, but not limited to.

Variations of each step in the Reaction Schemes above may include one or multiple synthetic steps. Isolation and purification of the product may be achieved by standard procedures known to those skilled in the art of organic chemistry.

In the process of preparing the compound represented by Formula I of the present invention, the compounds in the form of intermediates shown in Reaction Schemes 1 to 3 above are also included in the scope of the present invention.

Specifically, the present invention provides variants of GTM moieties in the form of GTM-RG¹, GTM-Linker-RG³ or GTM-Linker 1-RG^{2b}, and provides variants of ULM moieties in the form of ULM-RG⁴, RG^{3a}-Linker 2-ULM or RG²-Linker-ULM.

Use of GPX4 Protein Degradation-Inducing Compound

In one aspect, the present invention provides a composition for degrading GPX4 protein comprising a compound represented by Formula I, a stereoisomer thereof or a pharmaceutically acceptable salt thereof.

The present inventors first discovered the idea that the therapeutic efficacy of ferroptosis-related diseases, including cancer, could be achieved by inducing the degradation of GPX4 protein through the PROTAC compound represented by Formula I, and confirmed this idea by experiments, upon considering that at the time of knockdown of GPX4 protein in cells, the ferroptosis pathway is activated, the sensitivity to GPX4 inhibitors is greatly increased, and cell death is induced (document[Yang, Wan Seok, et al.], and the like).

As shown in FIG. 1, the compound represented by Formula I according to the present invention may recruit the GPX4 protein, which is a target protein, to the E3 ubiquitin ligase and induce ubiquitination of the GPX4 protein, thereby inducing degradation of the GPX4 protein by the ubiquitin-proteasome system (UPS) in cells. As a result of treating cancer cells with the compounds according to Examples of the present invention, which represent the compound of Formula I, it was confirmed that GPX4 protein was effectively degraded (see Experimental Example). Therefore, the compound represented by Formula I of the present invention may be usefully employed to induce degradation of GPX4 protein.

In an embodiment, the composition for degrading GPX4 protein may be administered to mammals, including humans, to degrade GPX4 protein. In this case, the composition may be a pharmaceutical composition further comprising at least one type of pharmaceutically acceptable carrier.

The compound represented by Formula I of the present invention may induce degradation of GPX4 protein, thereby making it possible to be usefully employed for preventing or treating GPX4-related diseases. In the present invention, GPX4-related disease refers to a condition or disease of which onset or progression is able to be inhibited, reduced, or alleviated by inhibiting the activity or inducing the degradation of GPX4 protein in the body. In an embodiment, the GPX4-related disease is a ferroptosis-related disease.

In the present invention, ferroptosis is a form of cell death, characterized by being mediated by iron and including the production of reactive oxygen species characterized in part by lipid peroxidation, and has the same meaning as that known in the art to which the present invention belongs.

In the present invention, the ferroptosis-related disease is a disease of which onset or progression is able to be inhibited, reduced, or alleviated by inducing, promoting, or activating intracellular ferroptosis, and may be cancer, lipid peroxidation-related degenerative diseases, excitotoxic diseases, neurodegenerative diseases, non-apoptotic regulated cell-death diseases, wasting- or necrosis-related diseases, addiction-related diseases, or infectious diseases, but is not limited to. In an embodiment, the ferroptosis-related disease is cancer.

In the present invention, the ferroptosis-related cancer may be a solid cancer or a hematological cancer. The solid cancer may be at least one selected from the group consisting of lung cancer (for example, non-small cell lung cancer), colorectal cancer, pancreatic cancer, kidney cancer, prostate cancer, breast cancer, ovarian cancer, stomach cancer, liver cancer, adrenal cortical cancer, anal cancer, cholangiocarcinoma, bladder cancer, bone cancer, glioma, astrocytoma, neuroblastoma, cervical cancer, endometrial cancer, esophageal cancer, head and neck cancer, intestinal cancer, oral cancer, salivary gland cancer, skin cancer (for example, malignant melanoma), testicular cancer, throat cancer, thyroid cancer, uterine cancer, vaginal cancer, sarcoma, and soft tissue carcinoma, but not limited to. The hematological cancer may be at least one selected from the group consisting of acute lymphoblastic leukemia (ALL), acute myeloid leukemia (AML), Hodgkin's lymphoma, non-Hodgkin's lymphoma (for example, diffuse large B-cell lymphoma), Burkitt's lymphoma, chronic lymphocytic leukemia (CLL), chronic myelogenous leukemia (CML), and multiple myeloma, but is not limited thereto.

In an embodiment, the ferroptosis-related cancer is a cancer with an activated or oncogenic RAS mutation. The RAS may be K-RAS, H-RAS or N-RAS.

In an embodiment, the ferroptosis-related cancer is a cancer that is resistant to chemotherapeutic agents or ionizing radiation. The chemotherapeutic agent may be an anti-cancer drug such as cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinplastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib, or a combination thereof, but is not limited thereto.

The pharmaceutical composition of the present invention may be a combination composition further comprising at least one type of drug of the same or similar class capable of helping treat, alleviate, delay, inhibit, or prevent GPX4-related diseases. In an embodiment, the at least one type of drug may be a ferroptosis-inducing drug.

The GPX4-related diseases, ferroptosis-related diseases and ferroptosis-inducing drugs according to the present invention may refer documents known in the art to which the present invention belongs, such as the references described herein, the entire contents of which are incorporated herein by reference.

The pharmaceutical composition of the present invention may be formulated through conventional methods in the field of pharmaceuticals, and may be formulated in various forms depending on specific types of GPX4-related diseases and drug components in combination therewith.

The pharmaceutical composition of the present disclosure may be administered orally or parenterally (for example, intravenous, subcutaneous, intraperitoneal or topical application) according to the desired method, and the dosage varies depending on the patient's weight, age, sex, health condition, diet, administration time, administration method, excretion rate, and severity of the disease, and the like.

In still another embodiment, the composition for degrading GPX4 protein may be treated in a sample in vitro to degrade GPX4 protein in the sample. The sample may be cells, cell cultures, body fluids or tissues of mammals including humans, and may be used for diagnostic or therapeutic purposes.

### Advantageous Effects

The compound of the present invention exhibits an effect of inducing degradation of GPX4 protein in cells, and thus may be usefully employed for preventing or treating GPX4-related diseases or ferroptosis-related diseases.

### DESCRIPTION OF DRAWINGS

FIG. 1 shows the principle that the bifunctional compound (Proteolysis Targeting Chimeras; PROTAC) according to the present invention recruits the target protein, GPX4, to an E3 ubiquitin ligase and then induces ubiquitination, thus leading to degradation; and

FIG. 2 shows a binding structure of CRBN and CRBN E3 ubiquitin ligase binding moiety (ULM).

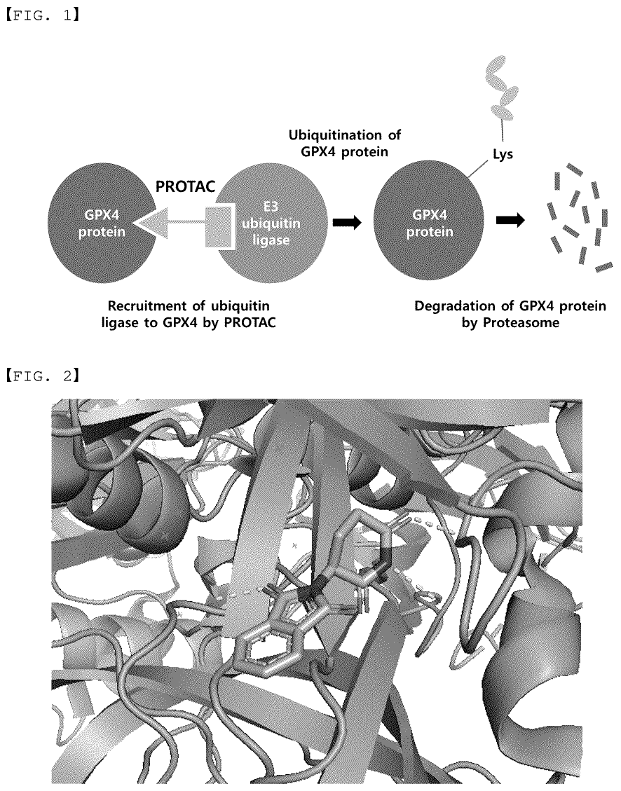

### BEST MODE

Hereinafter, the constitution and effects of the present disclosure will be described in more detail through Examples and Experimental Examples. These Examples and Experimental Examples are only provided for illustrating the present disclosure, but the scope of the present disclosure is not limited by these Examples. All documents cited throughout the present application are hereby expressly incorporated herein by reference in their entirety.

As the best mode for carrying out the present invention, provided are synthetic methods for compounds 1 to 5 listed in Table below.

TABLE 1

| Compound | Structure |
|---|---|
| 1 | 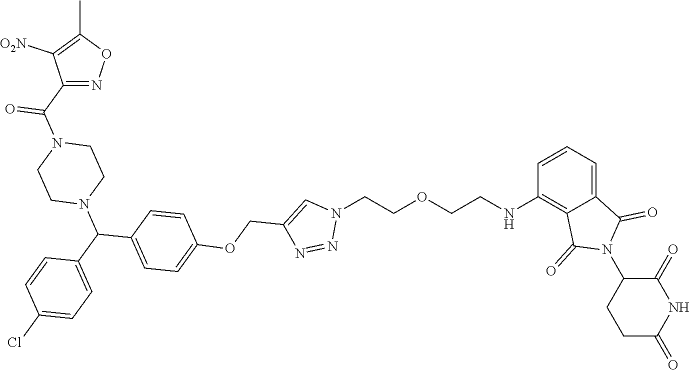 [SMILES: Cc1onc(C(=O)N2CCN(C(c3ccc(Cl)cc3)c3ccc(OCc4cn(CCOCCNc5cccc6c5C(=O)N(C5CCC(=O)NC5=O)C6=O)nn4)cc3)CC2)c1[N+](=O)[O-]] |
| 2 | 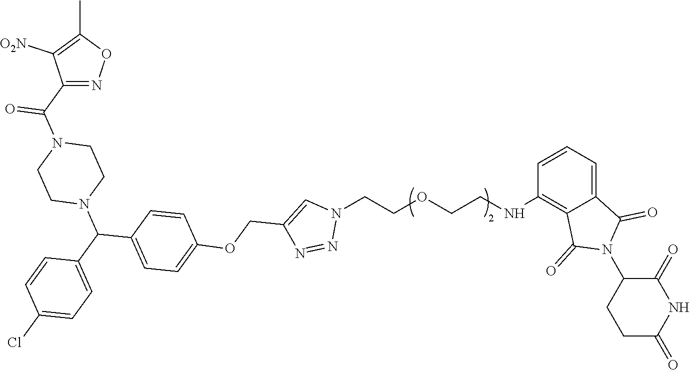 |
| 3 | 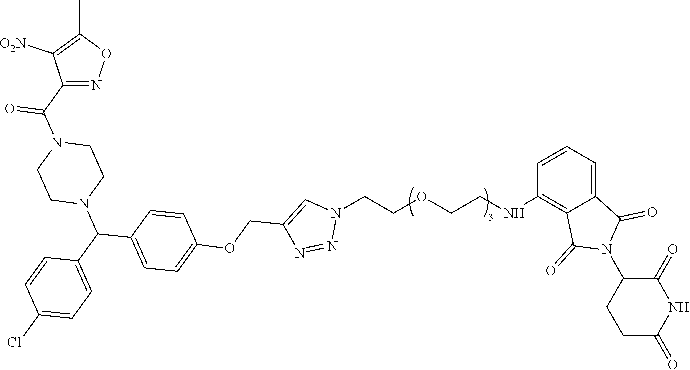 |
| 4 | 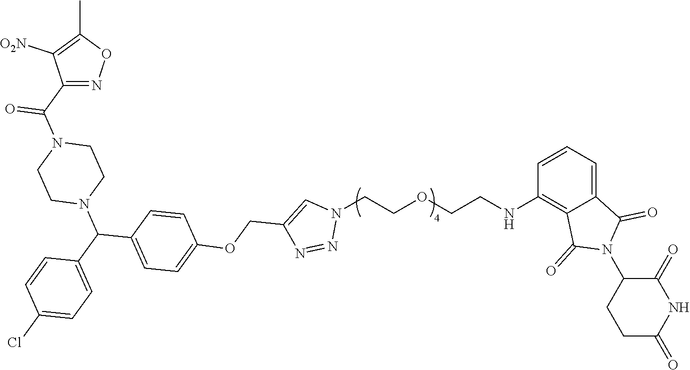 |
| 5 | 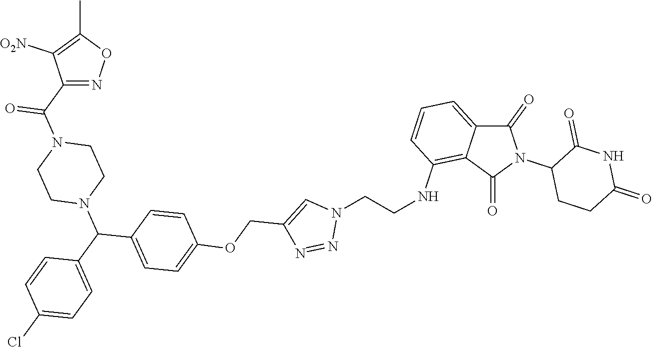 [SMILES: Cc1onc(C(=O)N2CCN(C(c3ccc(Cl)cc3)c3ccc(OCc4cn(CCNc5cccc6c5C(=O)N(C5CCC(=O)NC5=O)C6=O)nn4)cc3)CC2)c1[N+](=O)[O-]] |

The names of the compounds 1 to 5 are as follows.
4-((2-(2-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)ethoxy)ethyl)amino)-2-(2,6-dioxopiperidin-3-yl)isoindoline-1,3-dione (Compound 1);
4-((2-(2-(2-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)ethoxy)ethoxy)ethyl)amino)-2-(2,6-dioxopiperidin-3-yl)isoindoline-1,3-dione (Compound 2);
4-((2-(2-(2-(2-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)ethoxy)ethoxy)ethoxy)ethyl)amino)-2-(2,6-dioxopiperidin-3-yl)isoindoline-1,3-dione (Compound 3);
4-((14-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)-3,6,9,12-tetraoxatetradecyl)amino)-2-(2,6-dioxopiperidin-3-yl)isoindoline-1,3-dione (Compound 4); and
4-((2-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)ethyl)amino)-2-(2,6-dioxopiperidin-3-yl)isoindoline-1,3-dione (Compound 5).

The following example compounds according to Examples of the present invention were purified and structurally analyzed according to the methods below.

Analytical instrument
LCMS: Shimadzu LCMS-2020
NMR: BRUKER AVANCE III/400 MHz
HPLC: Agilent 1290 LC

LCMS Analysis Method

LCMS data were recorded by Shimadzu LCMS-2020 equipped with an electron spray ionization device. 0.0375% TFA in water (solvent A) and 0.01875% TFA in acetonitrile (solvent B) were used as mobile phases. As a column, Kinetex EVO C18 (2.1*30)mm, 5 μm was used.

HPLC Analysis Method

For HPLC, Agilent 1290 LC was used, and 0.0375% TFA in water (solvent A) and 0.01875% TFA in acetonitrile (solvent B) were used as mobile phases. As a column, Eclipse plus C18 (4.6*150)mm, 3.5 μm was used.

NMR Analysis Method

¹H NMR spectra were recorded with Bruker AVANCE III 400 MHz/5 mm Probe (HBO).

### <Example 1> Synthesis of 4-((2-(2-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)ethoxy)ethyl)amino)-2-(2,6-dioxopiperidin-3-yl)isoindoline-1,3-dione (Compound 1)

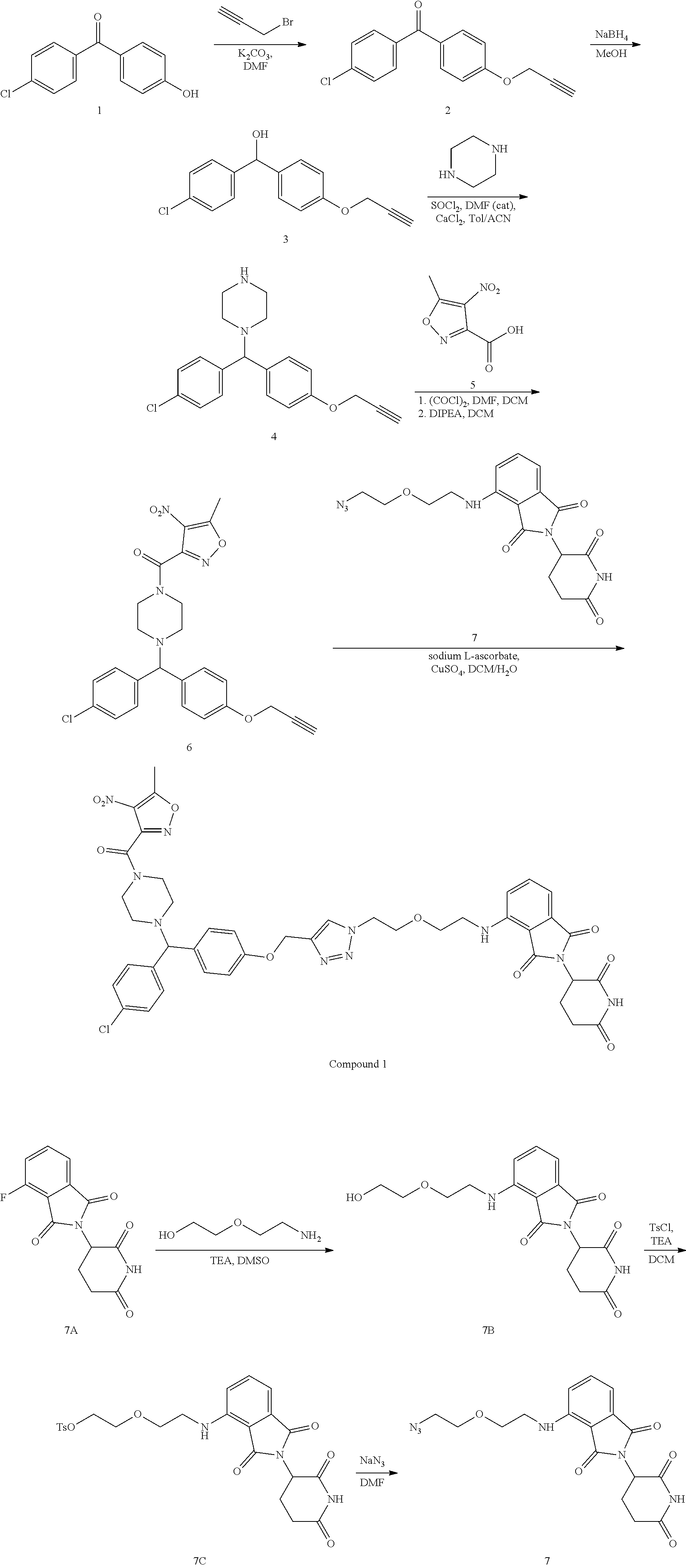

### Step 1. Synthesis of 2-(2,6-dioxopiperidin-3-yl)-4-((2-(2-hydroxyethoxy)ethyl)amino)isoindoline-1,3-dione (7B)

To a solution of 2-(2,6-dioxopiperidin-3-yl)-4-fluoroisoindoline-1,3-dione (20 g, 72.41 mmol), 2-(2-aminoethoxy)ethanol (9.90 g, 94.13 mmol, 9.42 mL) in DMSO (200 mL) was added TEA (21.98 g, 217.22 mmol, 30.23 mL) and heated at 80° C. for 12 h. LCMS showed the desired mass was detected. The mixture was quenched by addition of H₂O (400 mL) at 25° C., and then extracted with EtOAc (100 mL×6). The organic layers were washed with Brine (200 mL), dried over Na₂SO₄, filtered and concentrated under reduced pressure. The residue was purified by flash silica gel chromatography (Biotage, 80 g SepaFlash® Silica Flash Column, Eluent of 30-100% Ethyl acetate/Petroleum ether gradient @ 80 mL/min). The title compound (4.03 g, 11.15 mmol, 15.40% yield) was obtained as a yellow solid. MS(M+H)⁺=362.1.

### Step 2. Synthesis of 2-(2-((2-(2,6-dioxopiperidin-3-yl)-1,3-dioxoisoindolin-4-yl)amino)ethoxy)ethyl 4-methylbenzenesulfonate (7C)

To a solution of 2-(2,6-dioxopiperidin-3-yl)-4-((2-(2-hydroxyethoxy)ethyl)amino)isoindoline-1,3-dione (4.03 g, 11.15 mmol) in DCM (20 mL) were added TEA (1.69 g, 16.73 mmol, 2.33 mL) and TsCl (2.55 g, 13.38 mmol) and stirred at 25° C. for 12 h. LCMS showed the desired mass was detected. The mixture was diluted with H₂O (50 mL) and extracted with DCM (80 mL×2). The organic layers were washed with Brine (50 mL), dried over Na₂SO₄, filtered and concentrated under reduced pressure. The residue was purified by flash silica gel chromatography (Biotage, 40 g SepaFlash® Silica Flash Column, Eluent of 30-100% Ethyl acetate/Petroleum ether gradient @ 60 mL/min). The title compound (4.51 g, 8.07 mmol, 72.40% yield, 92.3% purity) was obtained as a yellow solid. MS(M+H)⁺=516.0.

### Step 3. Synthesis of 4-((2-(2-azidoethoxy)ethyl)amino)-2-(2,6-dioxopiperidin-3-yl)isoindoline-1,3-dione (7)

To a solution of 2-(2-((2-(2,6-dioxopiperidin-3-yl)-1,3-dioxoisoindolin-4-yl)amino)ethoxy)ethyl 4-methylbenzenesulfonate (1 g, 1.94 mmol) in DMF (10 mL) was added NaN₃ (252.20 mg, 3.88 mmol) and stirred at 80° C. for 16 h under N₂ atmosphere. LCMS showed the starting material was consumed completely and 98% of the desired mass was detected. H₂O (80 mL) was poured into the mixture slowly, then extracted with EtOAc (50 mL×3). The organic layers were concentrated under reduced pressure. The title compound (800 mg, crude) was obtained as a yellow solid, which was used in the next step without any further purification. MS(M+H)⁺=387.2.

### Step 4. Synthesis of (4-chlorophenyl) (4-(prop-2-yn-1-yloxy)phenyl)methanone (2)

To a solution of (4-chlorophenyl) (4-hydroxyphenyl)methanone (16.5 g, 70.92 mmol) and 3-bromoprop-1-yne (12.65 g, 85.10 mmol, 9.17 mL, 80% purity) in DMF (300 mL) was added K₂CO₃ (19.60 g, 141.84 mmol) and stirred at 20° C. for 12 h. TLC showed that the reaction was completed, then H₂O (300 mL) was poured into the mixture and filtered. The filter cake was collected and concentrated under reduced pressure to obtain the title compound (18.5 g, 63.56 mmol, 89.62% yield, 93% purity) as a white solid. (M+H)⁺=271.0.

### Step 5. Synthesis of (4-chlorophenyl) (4-(prop-2-yn-1-yloxy)phenyl)methanol (3)

To a solution of (4-chlorophenyl) (4-(prop-2-yn-1-yloxy)phenyl)methanone (18.5 g, 68.34 mmol) in MeOH (200 mL) was added NaBH₄ (1.6 g, 42.29 mmol) and stirred at 20° C. for 12 h. LCMS showed that the reaction was completed, then HCl (200 mL, 0.01 M) was added, concentrated under reduced pressure to remove methanol, and the mixture was extracted with EtOAc (200 mL×2). The organic layers were washed with H₂O (200 mL) and concentrated under reduced pressure to obtain the title compound (18 g, 66.00 mmol, 96.58% yield) as a yellow solid. MS(M+H)⁺=273.1.

### Step 6. Synthesis of 1-((4-chlorophenyl)(4-(prop-2-yn-1-yloxy)phenyl)methyl)piperazine (4)

To a solution of (4-chlorophenyl) (4-(prop-2-yn-1-yloxy)phenyl)methanol (5 g, 18.33 mmol) in Toluene (100 mL) was added CaCl₂) (20.35 g, 183.33 mmol), then added SOCl₂ (21.81 g, 183.33 mmol, 13.30 mL) and DMF (134.00 mg, 1.83 mmol, 141.05 μL) and stirred at 80° C. for 12 h. The mixture was concentrated and the residue was dissolved in ACN (100 mL), then piperazine (15.79 g, 183.33 mmol) was added and stirred at 80° C. for 4 h. LCMS showed that the reaction was completed, then the mixture was filtered. The filtrate was concentrated and the residue was purified by silica gel chromatography to obtain the title compound (3.7 g, 10.86 mmol, 59.21% yield) as a yellow oil. MS(M+H)⁺=341.2.

### Step 7. Synthesis of (4-((4-chlorophenyl) (4-(prop-2-yn-1-yloxy)phenyl)methyl)piperazin-1-yl) (5-methyl-4-nitroisoxazol-3-yl)methanone (6)

To a solution of 5-methyl-4-nitro-isoxazole-3-carboxylic acid (757.36 mg, 4.40 mmol) in DCM (10 mL) were added oxalyl chloride (558.58 mg, 4.40 mmol, 385.23 μL) and DMF (21.44 mg, 293.39 μmol, 22.57 μL) and stirred at 20° C. for 2 h under N₂ atmosphere. The mixture was concentrated to obtain 5-methyl-4-nitroisoxazole-3-carbonyl chloride, dissolved in DCM (10 mL), and then 1-((4-chlorophenyl) (4-(prop-2-yn-1-yloxy)phenyl)methyl)piperazine (1 g, 2.93 mmol) and DIPEA (758.37 mg, 5.87 mmol, 1.02 mL) were added at 0° C., then stirred at 20° C. for 2 h. TLC showed that the reaction was completed. The mixture was concentrated and purified by silica gel column to obtain the title compound (0.9 g, 1.69 mmol, 57.64% yield, 93% purity) as a yellow solid. MS(M+Na)⁺=517.0.

### Step 8. Synthesis of 4-((2-(2-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)ethoxy)ethyl)amino)-2-(2,6-dioxopiperidin-3-yl)isoindoline-1,3-dione (Compound 1)

To a solution of (4-((4-chlorophenyl) (4-(prop-2-yn-1-yloxy)phenyl)methyl)piperazin-1-yl) (5-methyl-4-nitroisoxazol-3-yl)methanone (150 mg, 303.08 μmol), 4-((2-(2-azidoethoxy)ethyl)amino)-2-(2,6-dioxopiperidin-3-yl)isoindoline-1,3-dione (128.81 mg, 333.38 μmol) in DCM (3 mL) and H₂O (1 mL) were added CuSO₄ (58.05 mg, 363.69 μmol) and sodium L-ascorbate (78.05 mg, 394.00 μmol) and stirred at 20° C. for 1 h. TLC (Ethyl acetate) showed the reaction was completed, then H₂O (20 mL) was poured into the mixture and extracted with DCM (20 mL×3). The organic layers were combined, washed with brine (50 mL), dried over Na₂SO₄, filtered and concentrated under reduced pressure. The residue was purified by prep-TLC (Ethyl acetate) to obtain the title compound (30 mg, 32.68 μmol, 10.78% yield, 96% purity) as a yellow solid. MS(M+H)⁺=881.4.

¹H NMR (400 MHz, CDCl₃) δ=7.99 (s, 1H), 7.79 (s, 1H), 7.52-7.46 (m, 1H), 7.35-7.33 (m, 2H), 7.24 (br d, J=2.8 Hz, 4H), 7.11 (d, J=7.2 Hz, 1H), 6.89 (dd, J=8.8, 16.0 Hz, 3H), 6.46 (br t, J=5.6 Hz, 1H), 5.14 (s, 2H), 4.95-4.79 (m, 1H), 4.56 (t, J=4.8 Hz, 2H), 4.22 (s, 1H), 3.90-3.79 (m, 4H), 3.64 (t, J=5.2 Hz, 2H), 3.40-3.35 (m, 4H), 2.86 (s, 3H), 2.82-2.60 (m, 3H), 2.51 (br t, J=4.8 Hz, 2H), 2.36 (br d, J=4.8 Hz, 2H), 2.05-2.02 (m, 1H).

### <Example 2> Synthesis of 4-((2-(2-(2-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)ethoxy)ethoxy)ethyl)amino)-2-(2,6-dioxopiperidin-3-yl)isoindoline-1,3-dione (Compound 2)

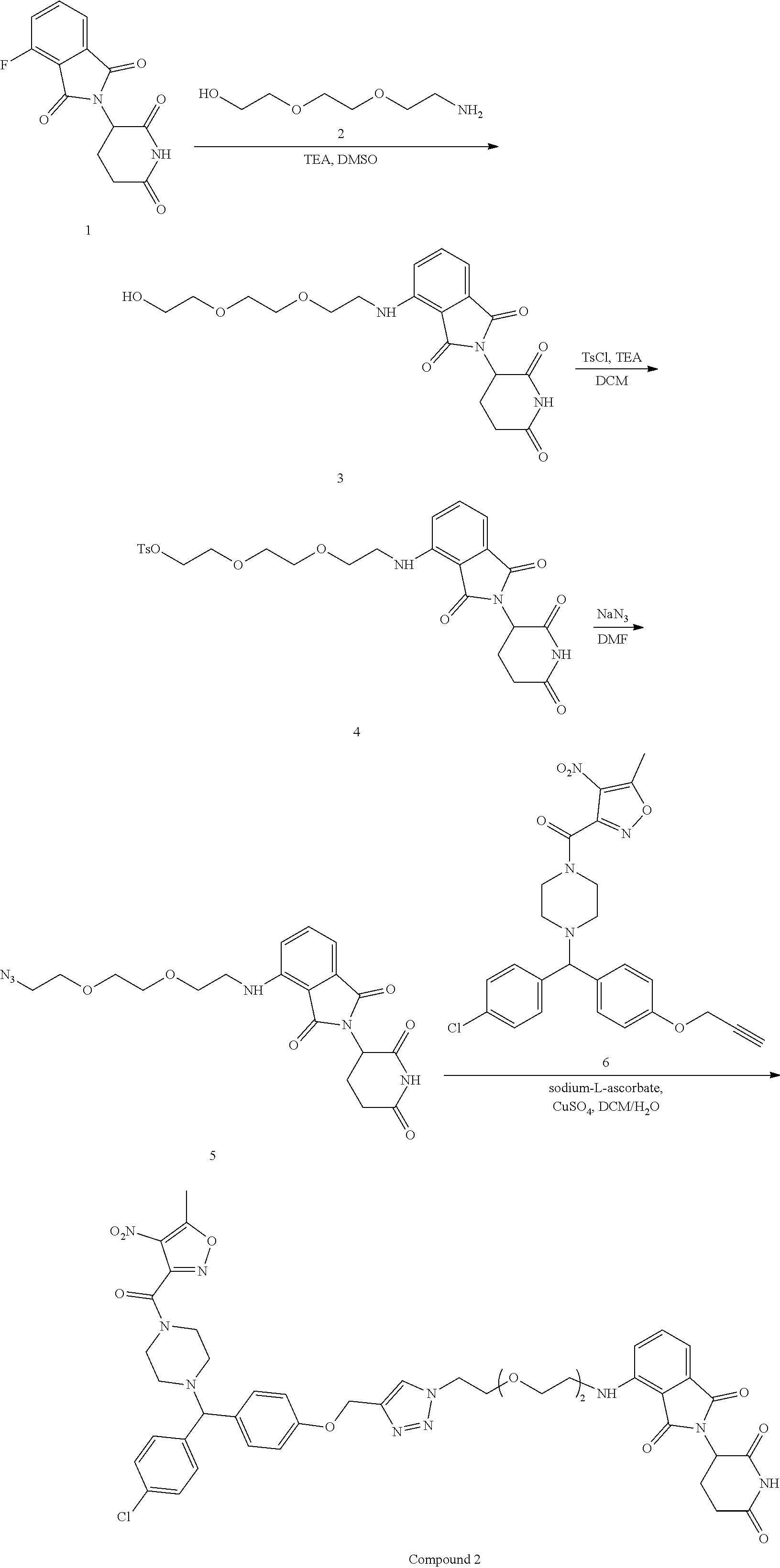

### Step 1. Synthesis of 2-(2,6-dioxopiperidin-3-yl)-4-((2-(2-(2-hydroxyethoxy)ethoxy)ethyl)amino)isoindoline-1,3-dione (3)

Two batches in parallel experiments: To a solution of 2-(2,6-dioxopiperidin-3-yl)-4-fluoroisoindoline-1,3-dione (5 g, 18.10 mmol) and 2-(2-(2-aminoethoxy)ethoxy)ethanol (2.97 g, 19.91 mmol) in DMSO (20 mL) was added TEA (3.66 g, 36.20 mmol, 5.04 mL) and stirred at 80° C. for 16 h under N₂ atmosphere. LCMS showed the starting material was consumed completely and 61% of the desired mass was detected. Two batches of the mixtures were diluted by adding H₂O (300 mL) and extracted with EtOAc (300 mL×5). The organic layers were washed with brine (400 mL×5), dried over Na₂SO₄, filtered and concentrated under reduced pressure. The title compound (7.4 g, crude) was obtained as a green oil, which was used in the next step without any further purification. MS(M+H)⁺=406.0.

### Step 2. Synthesis of 2-(2-(2-((2-(2,6-dioxopiperidin-3-yl)-1,3-dioxoisoindolin-4-yl)amino)ethoxy)ethoxy)ethyl 4-methylbenzenesulfonate (4)

To a solution of 2-(2,6-dioxopiperidin-3-yl)-4-((2-(2-(2-hydroxyethoxy)ethoxy)ethyl)amino)isoindoline-1,3-dione (7.3 g, 18.01 mmol) in DCM (40 mL) were added TEA (5.47 g, 54.02 mmol, 7.52 mL) and TsCl (6.18 g, 32.41 mmol) and stirred at 20° C. for 12 h. LCMS showed the starting material was consumed completely and 65% of the desired mass was detected. The mixture was concentrated under reduced pressure. The residue was purified by flash silica gel chromatography (ISCO®; 40 g SepaFlash® Silica Flash Column, Eluent of 40-80% Ethyl acetate/Petroleum ether gradient @ 100 mL/min). The title compound (5.8 g, 10.36 mmol, 57.56% yield) was obtained as a yellow solid. MS(M+H)⁺=560.1.

### Step 3. Synthesis of 4-((2-(2-(2-azidoethoxy)ethoxy)ethyl)amino)-2-(2,6-dioxopiperidin-3-yl)isoindoline-1,3-dione (5)

To a solution of 2-(2-(2-((2-(2,6-dioxopiperidin-3-yl)-1,3-dioxoisoindolin-4-yl)amino)ethoxy)ethoxy)ethyl 4-methylbenzenesulfonate (1 g, 1.79 mmol) in DMF (10 mL) was added NaN₃ (151.03 mg, 2.32 mmol) and stirred at 80° C. for 16 h. LCMS showed the starting material was consumed completely and 83% of the desired mass was detected. H₂O (100 mL) was poured into the mixture slowly, then extracted with EtOAc (80 mL×3). The organic layers were washed with brine (100 mL×5), dried over Na₂SO₄, filtered and concentrated under reduced pressure. The title compound (820 mg, crude) was obtained as a yellow solid, which was used in the next step without any further purification. MS(M+H)⁺=431.0.

### Step 4. Synthesis of 4-((2-(2-(2-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)ethoxy)ethoxy)ethyl)amino)-2-(2,6-dioxopiperidin-3-yl)isoindoline-1,3-dione (Compound 2)

To a solution of (4-((4-chlorophenyl) (4-(prop-2-yn-1-yloxy)phenyl)methyl)piperazin-1-yl) (5-methyl-4-nitroisoxazol-3-yl)methanone (150 mg, 303.08 μmol) and 4-((2-(2-(2-azidoethoxy)ethoxy)ethyl)amino)-2-(2,6-dioxopiperidin-3-yl)isoindoline-1,3-dione (143.49 mg, 333.38 μmol) in DCM (3 mL) and H₂O (1 mL) were added CuSO₄ (58.05 mg, 363.69 μmol, 55.82 μL) and sodium L-ascorbate (78.05 mg, 394.00 μmol) and stirred at 20° C. for 1 h. TLC showed that the reaction was completed, then H₂O (20 mL) was poured into the mixture and extracted with DCM (20 mL×3). The organic layers were combined, washed with brine (50 mL), dried over Na₂SO₄, filtered and concentrated under reduced pressure. The residue was purified by prep-TLC to obtain the title compound (80 mg, 84.73 μmol, 27.96% yield, 98% purity) as a yellow solid. MS(M+H)⁺=925.5.

¹H NMR (400 MHz, CDCl₃) δ=8.21 (s, 1H), 7.80 (s, 1H), 7.44 (dd, J=7.4, 8.4 Hz, 1H), 7.35-7.27 (m, 2H), 7.26-7.24 (m, 4H), 7.07 (d, J=7.0 Hz, 1H), 6.90-6.87 (m, 3H), 6.48 (t, J=5.4 Hz, 1H), 5.10 (s, 2H), 4.91-4.86 (m, 1H), 4.55 (t, J=4.8 Hz, 2H), 4.21 (s, 1H), 3.89 (t, J=5.2 Hz, 2H), 3.83 (br d, J=3.6 Hz, 2H), 3.68-3.65 (m, 2H), 3.64-3.59 (m, 4H), 3.42 (q, J=5.4 Hz, 2H), 3.38-3.33 (m, 2H), 2.87-2.80 (m, 4H), 2.78-2.67 (m, 2H), 2.50 (t, J=4.8 Hz, 2H), 2.36 (br s, 2H), 2.14-2.06 (m, 1H).

### <Example 3> Synthesis of 4-((2-(2-(2-(2-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)ethoxy)ethoxy)ethoxy)ethyl)amino)-2-(2,6-dioxopiperidin-3-yl)isoindoline-1,3-dione (Compound 3)

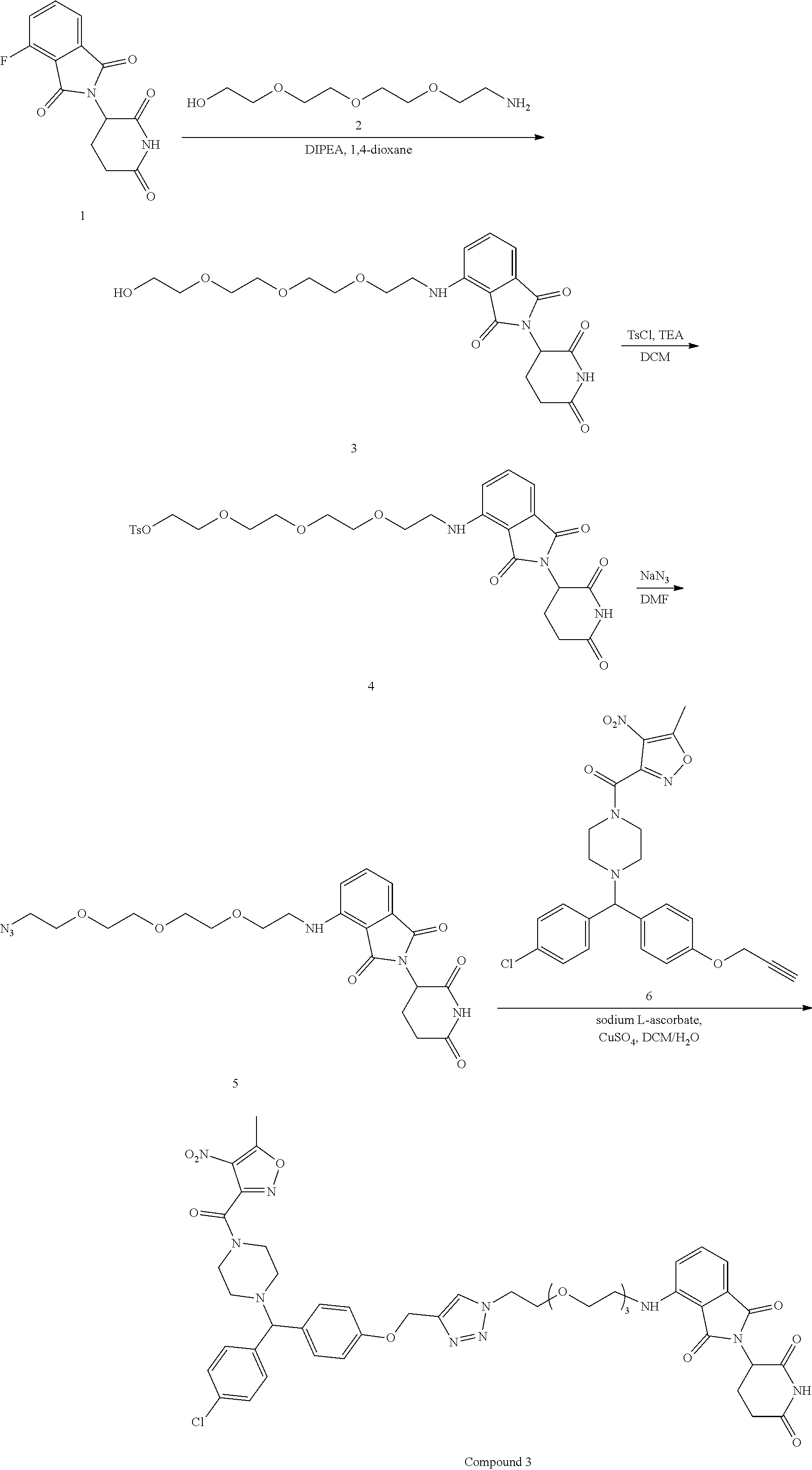

### Step 1. Synthesis of 2-(2,6-dioxopiperidin-3-yl)-4-((2-(2-(2-(2-hydroxyethoxy)ethoxy)ethoxy)ethyl)amino)isoindoline-1,3-dione (3)

To a solution of 2-(2,6-dioxopiperidin-3-yl)-4-fluoroisoindoline-1,3-dione (7 g, 25.34 mmol) and 2-[2-[2-(2-aminoethoxy)ethoxy]ethoxy]ethanol (5.00 g, 25.85 mmol) in 1,4-dioxane (40 mL) was added DIPEA (8.19 g, 63.36 mmol, 11.04 mL) and stirred at 115° C. for 16 h. LCMS showed the starting material was consumed completely and 44% of the desired mass was detected. The mixture was concentrated under reduced pressure. The residue was purified by flash silica gel chromatography (ISCO®; 40 g SepaFlash® Silica Flash Column, Eluent of 50-100% Ethyl acetate/Petroleum ether gradient @ 100 mL/min). The title compound (4.5 g, 10.01 mmol, 39.51% yield) was obtained as a green oil. MS(M+H)⁺=450.1.

### Step 2. Synthesis of 2-(2-(2-(2-((2-(2,6-dioxopiperidin-3-yl)-1,3-dioxoisoindolin-4-yl)amino)ethoxy)ethoxy)ethoxy)ethyl 4-methylbenzenesulfonate (4)

To a solution of 2-(2,6-dioxopiperidin-3-yl)-4-((2-(2-(2-(2-hydroxyethoxy)ethoxy)ethoxy)ethyl)amino)isoindoline-1,3-dione (4.5 g, 10.01 mmol) in DCM (20 mL) were added TEA (2.53 g, 25.03 mmol, 3.48 mL) and TsCl (3.82 g, 20.02 mmol) and stirred at 20° C. for 16 h. LCMS showed 4% of the starting material remained and 88% of the desired mass was detected. The mixture was concentrated under reduced pressure. The residue was purified by flash silica gel chromatography (ISCO®; 40 g SepaFlash® Silica Flash Column, Eluent of 25-80% Ethyl acetate/Petroleum ether gradient @ 100 mL/min). The title compound (5.2 g, 8.61 mmol, 86.04% yield) was obtained as a yellow oil. MS(M+H)⁺=604.2.

### Step 3. Synthesis of 4-((2-(2-(2-(2-azidoethoxy)ethoxy)ethoxy)ethyl)amino)-2-(2,6-dioxopiperidin-3-yl)isoindoline-1,3-dione (5)

To a solution of 2-(2-(2-(2-((2-(2,6-dioxopiperidin-3-yl)-1,3-dioxoisoindolin-4-yl)amino)ethoxy)ethoxy)ethoxy)ethyl 4-methylbenzenesulfonate (1.3 g, 2.15 mmol) in DMF (15 mL) was added NaN₃ (210.01 mg, 3.23 mmol) and stirred at 80° C. for 16 h under N₂ atmosphere. LCMS showed the starting material was consumed completely and 96% of the desired mass was detected. H₂O (80 mL) was poured into the mixture slowly, then extracted with EtOAc (50 mL×3). The organic layers were washed with brine (80 mL×5), dried over Na₂SO₄, filtered and concentrated under reduced pressure. The title compound (840 mg, crude) was obtained as a yellow solid, which was used in the next step without any further purification. MS(M+H)⁺=475.0.

### Step 4. Synthesis of 4-((2-(2-(2-(2-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)ethoxy)ethoxy)ethoxy)ethyl)amino)-2-(2,6-dioxopiperidin-3-yl)isoindoline-1,3-dione (Compound 3)

To a solution of (4-((4-chlorophenyl) (4-(prop-2-yn-1-yloxy)phenyl)methyl)piperazin-1-yl) (5-methyl-4-nitroisoxazol-3-yl)methanone (150 mg, 303.08 μmol) and 4-((2-(2-(2-(2-azidoethoxy) ethoxy) ethoxy)ethyl)amino)-2-(2,6-dioxopiperidin-3-yl)isoindoline-1,3-dione (215.70 mg, 454.61 μmol) in DCM (3 mL) and H₂O (1 mL) were added CuSO₄ (58.05 mg, 363.69 μmol, 55.82 μL) and sodium L-ascorbate (78.05 mg, 394.00 μmol) and stirred at 20° C. for 1 h. TLC showed that the reaction was completed, then H₂O (20 mL) was poured into the mixture and extracted by DCM (20 mL×3). The organic layers were combined, washed with brine (50 mL), dried over Na₂SO₄, filtered and concentrated under reduced pressure. The residue was purified by prep-TLC to obtain the title compound (40 mg, 40.03 μmol, 13.21% yield, 97% purity) as a yellow solid. MS(M+H)⁺=969.5.

¹H NMR (400 MHz, CDCl₃) δ=8.23 (br s, 1H), 7.80 (s, 1H), 7.47 (dd, J=7.4, 8.4 Hz, 1H), 7.36-7.32 (m, 2H), 7.26-7.24 (m, 4H), 7.10 (d, J=6.8 Hz, 1H), 6.92-6.90 (m, 3H), 6.46 (t, J=6.0 Hz, 1H), 5.14 (s, 2H), 4.92-4.88 (m, 1H), 4.53 (t, J=5.2 Hz, 2H), 4.22 (s, 1H), 3.88-3.82 (m, 4H), 3.66 (t, J=5.2 Hz, 2H), 3.64-3.60 (m, 4H), 3.59 (s, 4H), 3.42 (q, J=5.4 Hz, 2H), 3.38-3.33 (m, 2H), 2.88-2.83 (m, 4H), 2.79-2.72 (m, 2H), 2.51 (br t, J=5.0 Hz, 2H), 2.36 (br t, J=4.8 Hz, 2H), 2.13-2.08 (m, 1H).

### <Example 4> Synthesis of 4-((14-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)-3,6,9,12-tetraoxatetradecyl)amino)-2-(2,6-dioxopiperidin-3-yl)isoindoline-1,3-dione (Compound 4)

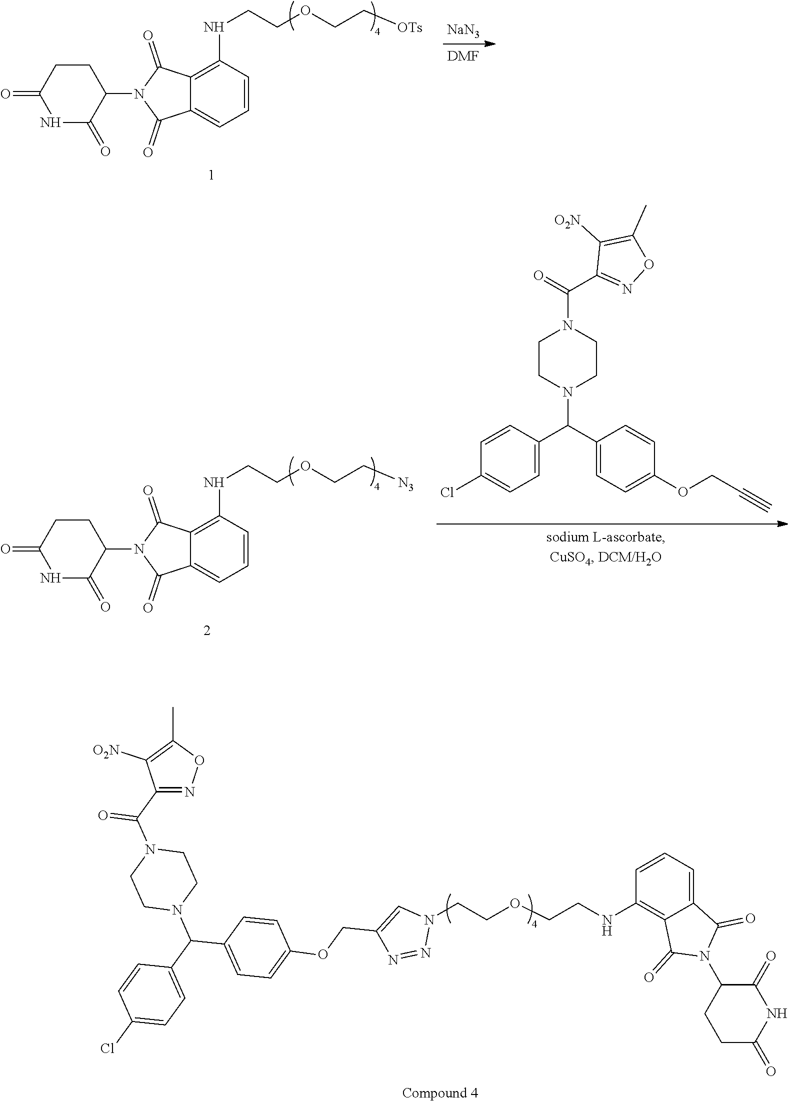

### Step 1. Synthesis of 4-((14-azido-3,6,9,12-tetraoxatetradecyl)amino)-2-(2,6-dioxopiperidin-3-yl)isoindoline-1,3-dione (2)

To a solution of 14-((2-(2,6-dioxopiperidin-3-yl)-1,3-dioxoisoindolin-4-yl)amino)-3,6,9,12-tetraoxatetradecyl 4-methylbenzenesulfonate (0.8 g, 1.24 mmol) in DMF (8 mL) was added NaN₃ (160.59 mg, 2.47 mmol) and stirred at 80° C. for 12 h under N₂ atmosphere. LCMS showed that the reaction was completed. H₂O (30 mL) was added into the mixture slowly, then extracted with EtOAc (50 mL×3). The organic layers were concentrated under reduced pressure to obtain the title compound (0.4 g, crude) as a yellow oil. MS(M+H)⁺=519.5.

### Step 2. Synthesis of 4-((14-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)-3,6,9,12-tetraoxatetradecyl)amino)-2-(2,6-dioxopiperidin-3-yl)isoindoline-1,3-dione (Compound 4)

To a solution of (4-((4-chlorophenyl) (4-(prop-2-yn-1-yloxy)phenyl)methyl)piperazin-1-yl) (5-methyl-4-nitroisoxazol-3-yl)methanone (150 mg, 303.08 μmol) and 4-((14-azido-3,6,9,12-tetraoxatetradecyl)amino)-2-(2,6-dioxopiperidin-3-yl)isoindoline-1,3-dione (0.4 g, crude) in DCM (3 mL) and H₂O (1 mL) were added CuSO₄ (58.05 mg, 363.70 μmol) and sodium L-ascorbate (78.05 mg, 394.00 μmol) and stirred at 20° C. for 1 h. LCMS showed that the reaction was completed, then H₂O (20 mL) was poured into the mixture and extracted with DCM (20 mL×3). The organic layers were combined, washed with brine (50 mL), dried over Na₂SO₄, filtered and concentrated under reduced pressure. The residue was purified by prep-TLC (Ethyl acetate) to obtain the title compound (89.3 mg, 81.95 μmol, 27.04% yield, 93% purity) as a yellow solid. MS(M+Na)⁺=1035.3.

¹H NMR (400 MHz, CDCl₃) δ=8.16 (br s, 1H), 7.74 (s, 1H), 7.44-7.38 (m, 1H), 7.27 (br d, J=8.0 Hz, 2H), 7.19-7.14 (m, 4H), 7.02 (d, J=7.1 Hz, 1H), 6.87-6.80 (m, 3H), 6.40 (br t, J=5.4 Hz, 1H), 5.07 (s, 2H), 4.82 (br dd, J=5.3, 12.0 Hz, 1H), 4.46 (t, J=5.0 Hz, 2H), 4.14 (s, 1H), 3.83-3.71 (m, 4H), 3.60 (br t, J=5.3 Hz, 2H), 3.57-3.48 (m, 14H), 3.36 (q, J=5.4 Hz, 2H), 3.28 (br s, 2H), 2.83-2.79 (m, 1H), 2.75 (br s, 1H), 2.73-2.63 (m, 2H), 2.43 (s, 2H), 2.29 (br s, 2H), 2.08-2.01 (m, 1H).

### <Example 5> Synthesis of 4-((2-(4-((4-((4-chlorophenyl)(4-(5-methyl-4-nitroisoxazole-3-carbonyl)piperazin-1-yl)methyl)phenoxy)methyl)-1H-1,2,3-triazol-1-yl)ethyl)amino)-2-(2,6-dioxopiperidin-3-yl)isoindoline-1,3-dione (Compound 5)

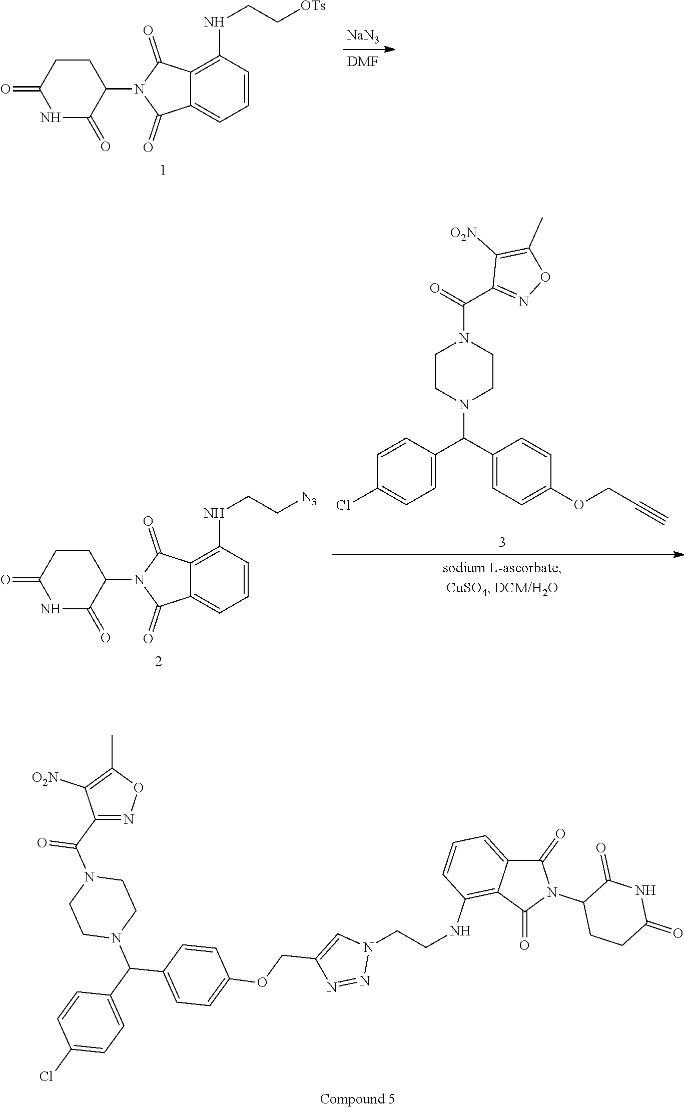

### Step 1. Synthesis of 4-((2-azidoethyl)amino)-2-(2,6-dioxopiperidin-3-yl)isoindoline-1,3-dione (2)

To a solution of 2-((2-(2,6-dioxopiperidin-3-yl)-1,3-dioxoisoindolin-4-yl)amino)ethyl 4-methylbenzenesulfonate (800 mg, 1.70 mmol) in DMF (10 mL) was added NaN₃ (132.37 mg, 2.04 mmol) and stirred at 80° C. for 16 h under N₂ atmosphere. LCMS showed that the starting material was consumed completely. H₂O (100 mL) was poured into the mixture slowly, then extracted with EtOAc (80 mL×3). The organic layers were washed with brine (100 mL×5), dried over Na₂SO₄, filtered and concentrated under reduced pressure. The title compound (600 mg, crude) was obtained as a yellow solid. MS(M+H)⁺=343.4.

### Step 2. Synthesis of 4-((2-(4-((4-((4-chlorophenyl) (4-(5-methyl-4-nitroisoxazole-3-carbonyl)piperazin-1-yl)methyl)phenoxy)methyl)-1H-1,2,3-triazol-1-yl)ethyl)amino)-2-(2,6-dioxopiperidin-3-yl)isoindoline-1,3-dione (Compound 5)

To a solution of (4-((4-chlorophenyl) (4-(prop-2-yn-1-yloxy)phenyl)methyl)piperazin-1-yl) (5-methyl-4-nitroisoxazol-3-yl)methanone (150 mg, 303.08 μmol) and 4-((2-azidoethyl)amino)-2-(2,6-dioxopiperidin-3-yl)isoindoline-1,3-dione (155.62 mg, 454.62 μmol) in DCM (3 mL) and H₂O (1 mL) were added CuSO₄ (58.05 mg, 363.70 μmol) and sodium L-ascorbate (78.05 mg, 394.00 μmol), and stirred at 20° C. for 1 h. LCMS showed that the reaction was completed, then H₂O (20 mL) was poured into the mixture and extracted with DCM (20 mL×3). The organic layers were combined, washed with brine (50 mL), dried over Na₂SO₄, filtered and concentrated under reduced pressure. The residue was purified by prep-TLC (Ethyl acetate) and the product was re-purified by prep-HPLC (column: Waters Xbridge 150*25 mm*5 um; mobile phase: [water (10 mM NH₄HCO₃)-ACN]; B %: 47%-77%, 10 min). The title compound (8.9 mg, 10.21 μmol, 3.37% yield, 96% purity) was obtained as a yellow solid. MS(M+H)⁺=837.1.

¹H NMR (400 MHz, DMSO-d₆) δ=11.07 (br s, 1H), 8.24 (br s, 1H), 7.54-7.41 (m, 3H), 7.33 (br d, J=18.0 Hz, 4H), 7.02 (br s, 2H), 6.95 (br s, 2H), 6.77 (br s, 1H), 5.06 (br s, 3H), 4.59 (br s, 2H), 4.38 (br s, 1H), 3.81 (br s, 2H), 3.69 (br s, 2H), 2.94-2.73 (m, 4H), 2.69-2.53 (m, 2H), 2.44-2.30 (m, 3H), 2.21 (br s, 2H), 2.00 (br d, J=4.4 Hz, 2H).

### Experimental Example

1. Culture of HeLa Cell Line

The HeLa cell line was purchased from Korea Cell Line Bank. The passage in cell culture was maintained around P25.

For cell counting, Thermo's cell counter (Catalog #AMQAX1000) and 0.4% trypan blue solution were used.

For cell culture, DMEM (Gibco, Cat. No. 11995-065; Lot. No. 2318854), FBS (Gibco, Cat. No. 16000044; Lot. No. 2351176P), penicillin/streptomycin (PS) (Gibco, Cat. No. 15140-122; Lot. No. 2321114), 100 mm culture plate (SPL, Cat. No. 20100), 12 well culture plate (SPL, Cat. No. 30012), PBS pH7.4 (Gibco, Cat. No. 10010-023; Lot. No. 2306390), Counting chamber (Hematocytometer) (Hirschmann, Cat. No. 8100204), and 0.4% trypan blue solution (DYNEBIO, Cat. No. CBT3710; Lot. No. 20190723) were used.

2. Treatment with Compounds of the Present Invention

3×10⁵ cells were seeded for each well of a 12 well plate (SPL), and the cells were cultured in the culture medium with a total volume of 1 mL.

The compound was completely dissolved in DMSO (Sigma, Cat. No. D2438; Lot. No. RNBK1809) and used in the experiment, wherein the concentration of DMSO treated in each well was unified to 0.1% and was treated by adding into cells.

Finally, in 24 hours after the treatment with the compound, the cells were washed with 0.5 mL of PBS pH7.4 (Gibco, Cat. No. 10010-023; Lot. No. 2306390), followed by treatment with 200 μL of TrypLE Express (Gibco, Cat. No. 12605-010; Lot. No. 2193192) to separate the cells. The isolated cells were neutralized by adding 1 mL of DMEM (Gibco, Cat. No. 11995-065; Lot. No. 2318854). The cells and the medium were separated using a centrifuge, and then only the cells were stored and used in subsequent Western blot experiments.

3. Western Blotting

For SDS-PAGE and Western blotting, M-PER lysis buffer (Thermo, Cat. no. 78503; Lot. no. WD319270), 100× protease inhibitor cocktail (Quartett, Cat. No. PPI1015; Lot no. PC050038424), Pierce™ BCA protein assay kit (ThermoScientific, Cat. No. 23225; Lot no. UC276876), albumin standard (ThermoScientific, Cat. No. 23209; Lot no. UB269561), 4-15% Mini-PROTEAN TGX stain-free gel (Bio-rad, Cat. No. 4568085; Lot no. L007041B), 10× Tris/Glycine/SDS buffer (Bio-rad, Cat. No. 1610732; Lot no. 10000044375B); Tris/Glycine buffer (Bio-rad, Cat. No. 1610734; Lot no. 64426132), 10× TBS (Bio-rad, Cat. No. 1706435; Lot no. 1000045140B), 10% Tween 20 solution (Cat. No. 1610781; Lot no. L004152B), Color protein standard broad range (NEB, Cat. No. P7719S; Lot no. 10040349), 4× Laemmli sample buffer (Bio-rad, Cat. No. 1610747; Lot no. L004133B), SuperBlock™ T20 (TBS) blocking buffer (ThermoScientific, Cat. No. 37536; Lot no. UC282578), 1M sodium azide solution (Sigma-Aldrich, Cat. No. 08591-1 mL-F; Lot no. BCBV4989), α-Rabbit pAb to Ms IgG (abcam, Cat. No. ab97046; Lot no. GR3252115-1), α-Rabbit IgG, HRP linked antibody (CST, Cat. no. 7074S; Lot no. 29), α-beta actin (sigma, Cat. No. A5316; Lot no. 029M4883V), α-GPX4 (Abcam, Cat. No. ab125066; Lot no. GR3229900-25), ECL™ Prime western blotting reagents (GE Healthcare, Cat. No. RPN2232; Lot no. 17001655), Ponceau S solution (Sigma-Aldrich, Cat. No. P7170; Lot no. SLBV4112), Difco™ Skim milk (BD, Cat. No. 232100; Lot no. 8346795), Acrylamide gel tank (Bio-Rad, Cat. No. 1658039), and PowerPac HC (Bio-Rad, Cat. No. 043BR80483) were used.

For cell lysis, a lysis buffer was added and cell debris was removed to obtain a cell lysate. Specifically, the cells were treated with 40 μL of 1× M-PER buffer containing a protease inhibitor and incubated on ice for 30 minutes. Then, the cells were centrifuged at 4° C. and 15,000 rpm for 15 minutes to obtain a cell lysate.

Then, a standard curve was obtained using the BCA assay, and the protein mass in the lysate was quantified by fitting to the standard curve. The mixture was incubated at 37° C. for 30 minutes using 20 μL of standard or sample solution, and 200 μL of BCA or Bradford reagent, and measured at 562 nm absorbance. Samples were prepared by adding 4× sample buffer to 15 μg per well.

SDS-PAGE was performed on a 4-15% Mini-PROTEAN TGX stain-free gel (15 well) by setting a running time of 100 min at 120 V. The samples were transferred to a Nitrocellulose membrane using a transfer buffer (prepared by mixing 10× Tris/Glycine buffer:methanol:DDW at a ratio of 1:2:7). The cells were stained with a Ponceau S solution, and blocked for 1 h with a blocking buffer (Thermo). The cells were washed with 1× TBS containing 0.05% Tween20, and reacted with, as primary antibodies in 1× TBS-T, anti-GPX4 (Abcam) antibody (1:2000) and anti-β-actin (sigma) antibody (1:5,000), at 4° C. for 16 hours. The cells were washed three times for 10 minutes with 1× TBS containing 0.05% Tween20, and reacted with, as secondary antibodies in 1× TBS-T, anti-mouse antibody (abcam) (1:5,000) and anti-rabbit antibody (CST), at room temperature for 1 h. Then, the cells were washed three times for 10 minutes with 1× TBS containing 0.05% Tween 20, and then detected with ECL working solution (1:1).

For analysis of the results, an image analyzer (GE) was used to obtain final blot data.

Together with the results, it was confirmed that the Example compounds of the present invention had excellent Dmax (60-80%) and DC₅₀ (0.5˜5 μM) values for the GPX4 protein, indicating that the GPX4 PROTAC compound of the present invention effectively degraded the target protein, GPX4.

1. A compound represented by the following Formula I, a stereoisomer thereof or a pharmaceutically acceptable salt thereof:
ULM-Linker-GTM [Formula I]
in Formula I above,
GTM is a Glutathione peroxidase 4 (GPX4) protein-binding moiety,
ULM is a CRBN E3 ubiquitin ligase binding moiety, and
Linker is a group that chemically connects ULM and GTM.

2. The compound, the stereoisomer thereof or the pharmaceutically acceptable salt thereof according to claim 1, wherein GTM is a GPX4 protein-binding moiety represented by the following Formula G-1: 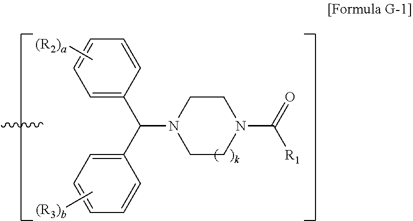
in Formula G-1 above,
R₂ and R₃ are each independently hydrogen, halogen, OH, NH₃, NO₂, CN, alkyl, alkene, alkyne, cycloalkyl, heterocyclyl, aryl or heteroaryl {wherein at least one hydrogen in R₁, R₂ and R₃ may each independently be optionally substituted};
k, a, and b are each independently an integer of 1 to 3; and [symbol]
represents that any one hydrogen or halogen in the Formula G-1 is substituted with a single bond and connected to the Linker by a covalent bond.

3. The compound, the stereoisomer thereof or the pharmaceutically acceptable salt thereof according to claim 2, wherein the Formula G-1 is a GPX4 protein-binding moiety defined as follows:
R₁ is selected 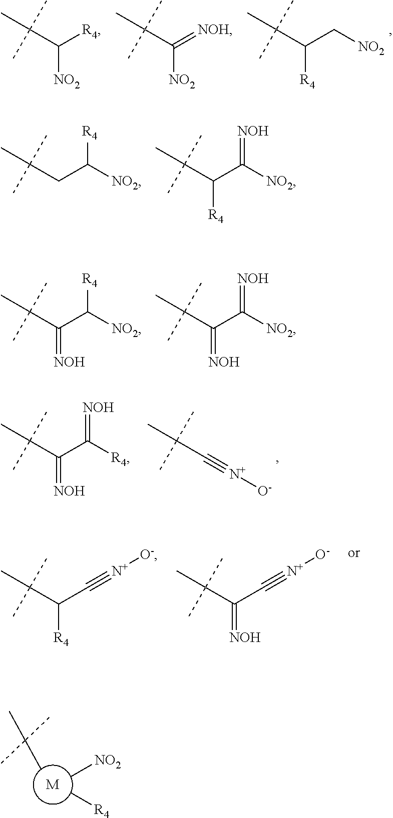
{wherein 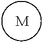
is 3-7 membered cycloalkyl, 4-8 membered heterocyclyl, phenyl or 4-8 membered heteroaryl};
R₂ and R₃ are each independently hydrogen, halogen, OH, NH₃, NO₂, CN, C₁₋₆alkyl, C₁₋₆ alkoxy, C₂₋₆alkenyl or C₂₋₆ alkynyl {wherein the C₁₋₆alkyl, C₁₋₆ alkoxy, C₂₋₆ alkenyl or C₂₋₆ alkynyl may be substituted with 1 to 3 halogens, OH, NH₃, NO₂ or CN};
R₄ is —(C₀₋₄ alkylene)-R₅, —(C₀₋₄ alkylene)-R_{L1}—(C₀₋₄alkylene)-R₅ or —(C₀₋₄alkylene)-R_{L1}—(C₀₋₄alkylene)-R_{L2}—(C₀₋₄alkylene)-R₅ {wherein at least one hydrogen in R₄ may each independently be substituted with halogen, C₁₋₃ alkyl, C₁₋₃ alkoxy, OH, NH₂, NO₂ or CN};
R₅ is hydrogen, halogen, OH, OCH₃, COH, COOH, CN, NH₂, NH(C₁₋₃alkyl), NCH₃(C₁₋₃alkyl), SO₂, C₁₋₆alkyl, C₂₋₆ alkenyl, C₂₋₆ alkynyl, C₃₋₈ cycloalkyl, 4-8 membered heterocyclyl, phenyl or 4-8 membered heteroaryl;
R_{L1} and R_{L2} are each independently —O—, —CO—, —COO—, —OCO—, —NH—, —N(C₁₋₃alkyl)-, —NHCO—, —N(C₁₋₃alkyl)CO—, —CONH—, —CON(C₁₋₃alkyl)- or —NHCONH—;
a, b and k are each independently 1 or 2; and [symbol]
represents that any one hydrogen or halogen in the Formula G-1 is substituted with a single bond and connected to the Linker by a covalent bond.

4. The compound, the stereoisomer thereof or the pharmaceutically acceptable salt thereof according to claim 3, wherein GTM is a GPX4 protein-binding moiety represented by the following Formula G-2: 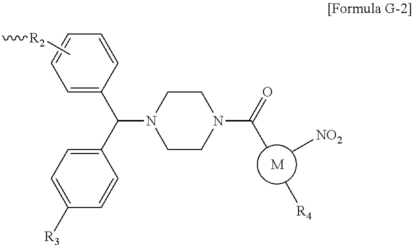
in Formula G-2 above, 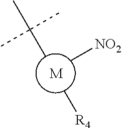
is selected from 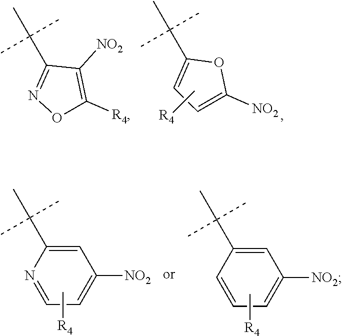
R₂ and R₃ are each independently hydrogen, halogen, OH, NH₃, C₁₋₆alkyl, C₁₋₆ alkoxy {wherein the C₁₋₆alkyl or C₁₋₆ alkoxy may be substituted with one halogen or CN};
hydrogen or halogen in R₂ is substituted with [symbol] {wherein [symbol] represents that it is linked to the Linker by a covalent bond};
R₄ is —(C₀₋₂alkylene)-R₅ or —(C₀₋₂alkylene)-R_{L1}—(C₀₋₂alkylene)-R₅ {wherein one hydrogen in R₄ may be substituted by halogen or OH};
R₅ is hydrogen, halogen, COOH, C₁₋₆alkyl, C₂₋₆ alkenyl, C₅₋₆cycloalkyl, 5-6 membered heterocyclyl, phenyl or 5-6 membered heteroaryl; and
R_{L1} is —O—, —CO—, —COO—, —OCO—, —NHCO— or —CONH—.

5. The compound, the stereoisomer thereof or the pharmaceutically acceptable salt thereof according to claim 1, Wherein ULM is a CRBN E3 ubiquitin ligase binding moiety represented by the following Formula A-1: 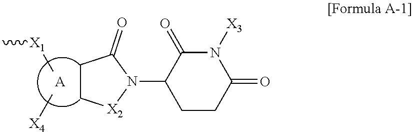
in Formula A-1 above, 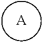
is a ring selected from 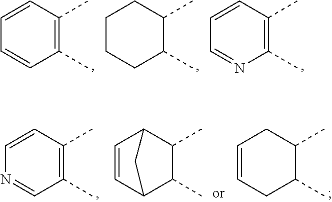
X₁ is a single bond, —CH₂—, —NH—, —O—, —CH₂CH₂—, —CC— —CO—, —COO—, —NHCO— or —CONH—;
X₂ is —CH₂—, —CH(C₁₋₄ alkyl)-, —NH—, —N(C₁₋₄ alkyl)-, —O—, —CO—, —CH₂—CH₂—, —NH—CH₂—, —NH—CH(C₁₋₄alkyl)-, —N═CH—, —N═C(C₁₋₄alkyl)- or —N═N—;
X₃ is hydrogen or C₁₋₄ alkyl;
X₄ is hydrogen, halogen, C₁₋₆alkyl, CN, NH₂, NO₂, OH, COH, COOH or CF₃; and [symbol]
represents that the Linker is covalently linked to the moiety represented by the Formula A-1 above.

6. The compound, the stereoisomer thereof or the pharmaceutically acceptable salt thereof according to claim 5, wherein ULM is a CRBN E3 ubiquitin ligase binding moiety represented by the following Formula A-2: 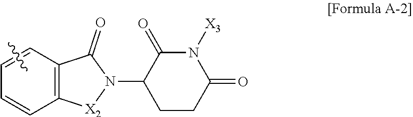
in Formula A-2 above,
X₂ is —CH₂—, —CH(C₁₋₄ alkyl)-, —CO— or —N═N—;
X₃ is hydrogen or C₁₋₃ alkyl; and [symbol]
represents that the Linker is covalently linked to the moiety represented by the Formula A-2 above.

7. The compound, the stereoisomer thereof or the pharmaceutically acceptable salt thereof according to claim 1, wherein the Linker is a chemical group represented by the following Formula L: 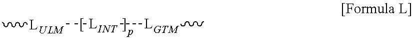
in Formula L above, [symbol]
and [symbol] are bonds;
L_{ULM} binds to the ULM moiety through [symbol] linked thereto;
L_{GTM} binds to the GTM moiety through [symbol] linked thereto,
L_{ULM}, L_{GTM}, and L_{INT} are each independently selected from a single bond, —CH₂—, —NH—, —O—, —S—, —SO—, —SO₂—, —CO—, —CH₂CH₂—, —CHCH—, —CC—, —CH₂CH₂O—, —OCH₂CH₂—, —CH₂CH₂S—, —SCH₂CH₂—, —COO—, —CONH—, —NHCO— or 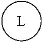
{wherein 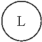
is cycloalkyl, heterocyclyl, aryl or heteroaryl};
L_{ULM}, L_{GTM}, and L_{INT} may each independently be substituted with at least one C₁₋₆ alkyl, C₃₋₈cycloalkyl, halogen, hydroxy, amine, nitro, cyano or haloalkyl; and
p is an integer from 1 to 30.

8. The compound, the stereoisomer thereof or the pharmaceutically acceptable salt thereof according to claim 1, wherein the compound is listed in Table below:

| Compound | Structure |
|---|---|
| 1 | 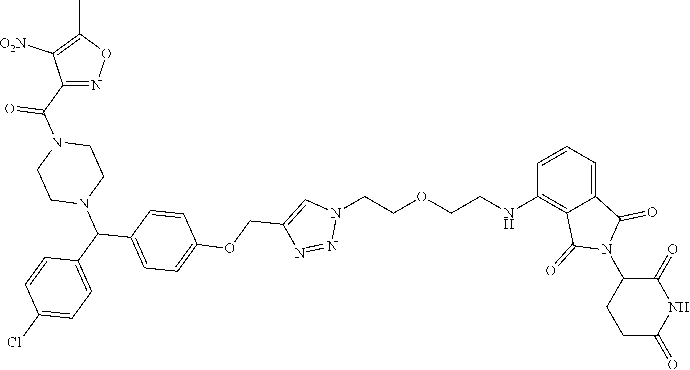 [SMILES: Cc1onc(C(=O)N2CCN(C(c3ccc(Cl)cc3)c3ccc(OCc4cn(CCOCCNc5cccc6c5C(=O)N(C5CCC(=O)NC5=O)C6=O)nn4)cc3)CC2)c1[N+](=O)[O-]] |
| 2 | 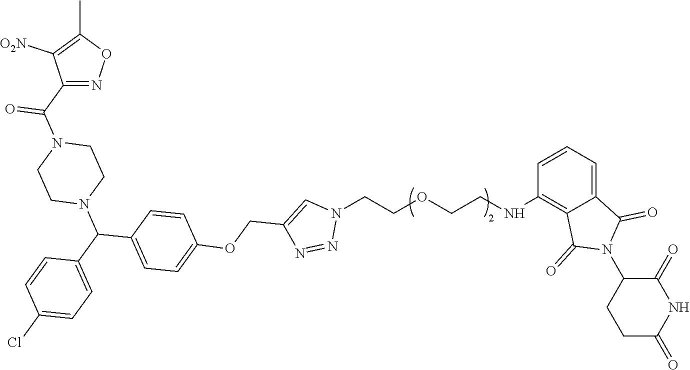 |
| 3 | 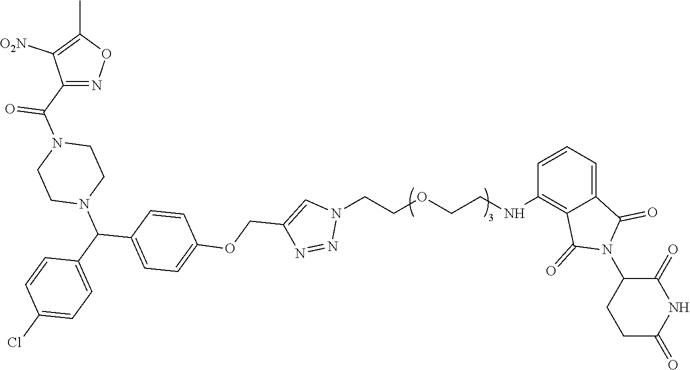 |
| 4 | 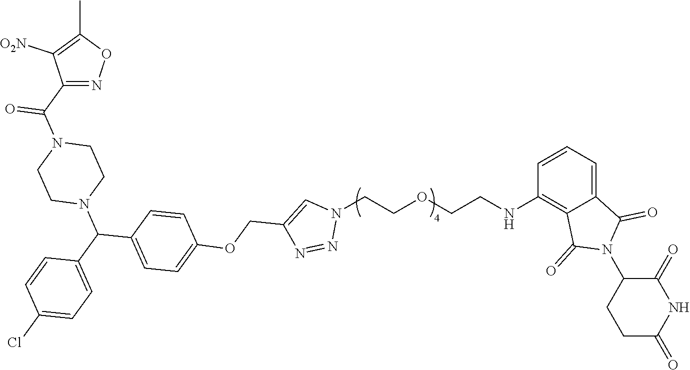 |
| 5 | 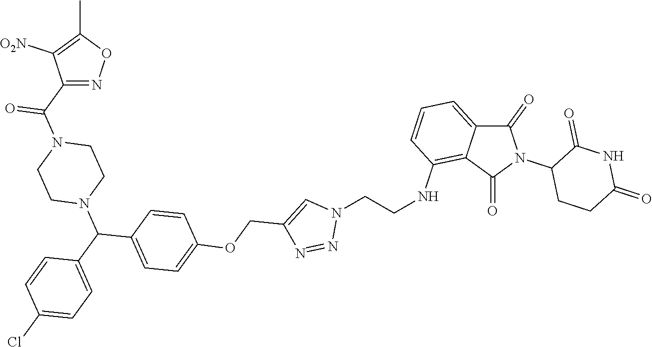 [SMILES: Cc1onc(C(=O)N2CCN(C(c3ccc(Cl)cc3)c3ccc(OCc4cn(CCNc5cccc6c5C(=O)N(C5CCC(=O)NC5=O)C6=O)nn4)cc3)CC2)c1[N+](=O)[O-]] |

9. A composition for degrading GPX4 protein comprising the compound, the stereoisomer thereof or the pharmaceutically acceptable salt thereof of claim 1.

10. The pharmaceutical composition according to claim 9, wherein the pharmaceutical composition further comprises at least one type of pharmaceutically acceptable carrier.

11. The pharmaceutical composition according to claim 9, wherein the pharmaceutical composition is for preventing or treating GPX4-related diseases.

12. The pharmaceutical composition according to claim 11, wherein the pharmaceutical composition is for preventing or treating ferroptosis-related diseases.

13. The pharmaceutical composition according to claim 12, wherein the pharmaceutical composition is for preventing or treating cancers.

14. A method for preventing or treating GPX4-related diseases comprising administering to patients a therapeutically effective amount of the compound of claim 1.
